(19) United States
(12) Patent Application Publication
(10) Pub. No.: US 2022/0412956 A1
(43) Pub. Date: Dec. 29, 2022

(54) METHODS FOR ASSESSING TOXICITY OF A COMPOUND

(71) Applicant: The Chinese University of Hong Kong, Shatin (CN)

(72) Inventors: Ellen Ngar Yun POON, Shatin (CN); Wai-Lung Billy NG, Tai Po (CN); Ka Shing Maxwell KWOK, Shatin (CN); Carrie LEE, Kau To Shan (CN)

(21) Appl. No.: 17/750,123

(22) Filed: May 20, 2022

Related U.S. Application Data
(60) Provisional application No. 63/191,782, filed on May 21, 2021.

(51) Int. Cl. G01N 33/50; C12N 9/14

(52) U.S. Cl. CPC G01N 33/5014; C12N 9/14

(57) ABSTRACT

The present invention provides methods for assessing a compound's potential toxicity.

### RELATED APPLICATIONS

This application claims priority to U.S. Provisional Patent Application No. 63/191,782, filed May 21, 2021, the contents of which are hereby incorporated by reference in the entirety for all purposes.

### BACKGROUND OF THE INVENTION

Infectious diseases are highly prevalent to the world's population. The current pandemic of COVID-19 is just such an example of an infective pathogen inflicting an exceptionally large loss in human lives as well as profound damages to social and economic stability of many countries. Nucleoside/nucleotide analogues constitute an important class of small molecular therapeutics for the treatment of infections. Mitochondrial toxicity is the main cause for the termination of clinical trials and for drug withdrawal post marketing. Drug-induced toxicity significantly impacts upon patient health and represents a financial burden to the pharmaceutical industry. Thus, there exists an urgent need to develop simple and effective methods for predicting and assessing mitochondrial toxicity of newly proposed drugs in therapeutic applications. The present invention fulfills this and related needs by providing new methods useful for predicting and detecting drug toxicity prior to performing more extensive tests in animals or humans.

### BRIEF SUMMARY OF THE INVENTION

The present invention resides in the discovery that the protein DRP1, encoded by the DNM1L gene, is a molecular target of therapeutics such as nucleotide/nucleoside analogues and mediates mitochondrial toxicity induced by the drugs. As such, a preliminary indication of cytotoxicity may be obtained by detecting interactions between the DRP1 protein and a proposed drug, which can in turn serve as a basis for additional and more accurate evaluation of cytotoxicity. Thus, in a first aspect, the present invention provides a method for assessing toxicity of a compound. The method comprises these steps: (1) detecting specific binding between the compound and DRP1 protein at its GTPase domain; and (2) determining the compound as likely toxic, e.g., the compound having 50% or high chances of possessing detectable cytotoxicity, especially against specific cell types of interest (e.g., cardiomyocytes). Upon such determination, the compound may be further tested to either confirm toxicity or eliminate suspected toxicity. Such further testing step may be performed by way of an in vitro cell-based cytotoxicity assay, for example, by directly exposing the cell type of interest (e.g., cardiomyocytes such as human pluripotent stem cell-derived cardiomyocytes or hPCS-CMs) to the compound in an effective amount or concentration for an appropriate amount of time and then determining whether a cytotoxic effect on the cells can be observed.

In some embodiments, step (1) of the method of this invention comprises simulating binding between the compound and the GTPase domain by molecular docking. In some embodiments, step (1) of the method comprises contacting the DRP1 protein or the GTPase domain of the DRP1 protein with the compound, for example, in an in vitro assay where the compound and the DRP1 protein or a fragment of the DRP1 protein comprising the GTPase domain or its GTPase domain are placed together under conditions permissible for their interaction. In some embodiments, the method is carried out in a cell-based assay, and step (1) comprises contacting a cell expressing the DRP1 protein or a fragment of the DRP1 protein comprising the GTPase domain or its GTPase domain with the compound. In some embodiments, the method further comprises, after step (2) when a test compound is deemed a likely cytotoxic agent, performing a cell-based toxicity assay to detect the compound's cytotoxicity to cells (especially a cell type of interest, e.g., human pluripotent stem cell-derived cardiomyocytes or hPCS-CMs), so as to confirm or eliminate the compound as a cytotoxic agent. In some embodiments, the cell-based toxicity assay is an in vitro hiPSC-CM cytotoxicity assay. In some embodiments, the method comprises measuring GTPase activity of the DRP1 protein in the presence and absence of the compound. In some embodiments, when an increase in the GTPase activity is detected in the presence of the compound, the compound is indicated as having toxicity. In some embodiments, the method further comprises, after step (2), measuring polymerization of the DRP1 protein with itself or with binding partners FIS1, MFF, MIEF, MiD49, MiD51 in the presence and absence of the compound. In some embodiments, an increase in the polymerization in the presence of the compound is detected and indicates toxicity of the compound. In some embodiments, the method further comprises, after step (2), measuring localization of the DRP1 protein in the mitochondria in the presence and absence of the compound. In some embodiments, an increase in the localization of DRP1 protein in the mitochondria is detected in the presence of the compound and indicates toxicity of the compound. In some embodiments, the method further comprises, after step (2), measuring DRP1 expression level in the presence and absence of the compound. In some embodiments, the expression level is DRP1 mRNA level. In some embodiments, the expression level is DRP1 protein level. In some embodiments, an increase in the DRP1 expression level, either at protein or mRNA level, is detected in the presence of the compound, which indicates toxicity of the compound. In some embodiments, the method further comprises, after step (2), monitoring morphology of mitochondria within the cell in the presence and absence of the compound. In some embodiments, increased mitochondria fragmentation is detected in the presence of the compound and indicates toxicity of the compound. In some embodiments, the method further comprises, after step (2), measuring redox potential of mitochondria within the cell in the presence and absence of the compound. In some embodiments, reduced mitochondria redox potential is detected in the presence of the compound and indicates toxicity of the compound. In some embodiments, the method further comprises, after step (2), measuring mRNA or protein level of one or more of ND1, NDS, COX6A2, and ATP6 within the cell in the presence and absence of the compound. In some embodiments, a decrease in the mRNA or protein level of one or more of ND1, ND5, COX6A2, and ATP6 is detected in the presence of the compound and indicates toxicity of the compound. In some embodiments, the cell used in the method is a cardiomyocyte (CM), and the method further comprises, after step (2), monitoring morphology of sarcomere within the cell in the presence and absence of the compound. In some embodiments, increased sarcomere disarray is detected in the presence of the compound, which indicates toxicity of the compound. In some embodiments, the cells used in the method of this invention are human pluripotent stem cell-derived cardiomyocytes (hPCS-CMs), which comprise both human embryonic stem cell-derived cardiomyocytes (hESC-CMs) and human induced pluripotent stem cell-derived cardiomyocytes (hiPSC-CM). While the present invention provides methods for testing toxicity of any compound as a potential therapeutic agent, in some cases, the compound being tested is a derivative of a nucleoside or nucleotide containing the core structure of a sugar (e.g., a 5-carbon sugar), a nucleobase, and optionally a phosphate group.

### BRIEF DESCRIPTION OF THE DRAWINGS

FIG. 1. Remdesivir induced mitochondrial dysfunction in hiPSC-CMs. Human iPSC-CMs were treated with indicated doses of remdesivir under normoxic or hypoxic conditions for 3 days. (A) Mitochondrial redox activity was measured using the PrestoBlue assay, n=11. (B) Fluorescence images of mitotracker dye staining (red) revealed disturbed mitochondrial organisation in hiPSC-CMs treated with remdesivir. Hoescht nuclear staining is in blue. The proportion of cells with elongated (asterick), punctate (arrowhead) and perinuclear (arrow) mitochondria were quantified, n=7 (C) Mitochondrial mass was measured with mitotracker dye (MTDR), n=4. (D) ROS level was measured with mitosox red dye, n=3. (E) Mitochondrial membrane potential (Δψm) was quantified using the TMRE mitochondrial dye, n=3. Statistical significance was calculated relative to control cells using the one-way ANOVA with Dunnett's multiple comparisons test for (A), (C-E), and two-way ANOVA with Sidak's multiple comparisons test for (B), *p<0.05, **p<0.01, ***p<0.001, ****p<0.0001.

FIG. 2. Remdesivir perturbed the expression of genes important for mitochondrial function. Human iPSC-CMs were treated with indicated doses of remdesivir under normoxic or hypoxic conditions for 3 days. The expression of selected genes were measured by qRT-PCR, normalised to B2M, n=5. Statistical significance was calculated using the one-way ANOVA with Dunnett's multiple comparisons relative to control cells *p<0.05, **p<0.01, ***p<0.001, ****p<0.0001.

FIG. 3. Remdesivir induced sarcomeric disarray in hiPSC-CMs. (A) Confocal images of hiPSC-CMs showing MLC2V-eGFP signal in green, Hoescht nuclear staining in blue. Representative images of 6 independent batches of cells are shown. Control hiPSC-CMs have densely-packed, striated MLC2V-eGFP signal, reflecting its localization to the thick filaments of sarcomeres, and was absent from the Z-disk and I-band. Sparse and truncated sarcomeres (arrowheads), patchy MLC2V-eGFP signal with no or poorly discernible organisation (arrows) could be detected in cells treated with high doses of remdesivir. (B) The proportion of cells with condensed nuclei was assessed among control cells and cells treated with the indicated doses of remdesivir. Statistical significance was examined using the one-way ANOVA test and no statistically significant difference was detected among the samples, n=8.

FIG. 4. Remdesivir induced persistent mitochondrial and structural abnormalities in hiPSC-CMs. (A) Human iPSC-CMs were treated with indicated doses of remdesivir under normoxic (blue) or hypoxic (yellow) conditions for 3 days, and allowed to recover in the absence of remdesivir for 3 more days under normoxic conditions. (B) Mitochondrial redox activity was measured using the PrestoBlue assay, n=8. (C) Mitochondrial morphology was assessed using mitotracker staining. Control cells mostly exhibited elongated mitochondria (asterick), the majority of remdesivir treated cells had punctate mitochondria (arrowhead). Perinuclear mitochondria (arrow) were enriched in cells treated with 12.5 mM of remdesivir, n=5. (D) Confocal images of hiPSC-CMs showing MLC2V-eGFP signal in green, Hoescht nuclear staining in blue. Sparse and truncated sarcomeres (arrowheads), patchy MLC2V-eGFP signal with no or poorly discernible organisation (arrows) could be detected in cells treated with remdesivir. Dotted arrow indicates a cell with brightly stained, condensed nuclei. (E) Quantification of cells with condensed nuclei, n=6. Statistical significance was calculated relative to control cells using the one-way ANOVA with Dunnett's multiple comparisons test for (B) and (E), and two-way ANOVA with Sidak's multiple comparisons test for (C),*p<0.05, **p<0.01, ***p<0.001, ****p<0.0001.

FIG. 5. An inhibitor of mitochondrial fission protected against remdesivir induced cardiotoxicity. (A) Human iPSC-CMs were treated with indicated doses of remdesivir under normoxic or hypoxic conditions. The mRNA expression of DRP1 was measured by qRT-PCR analysis, normalised to B2M, n=4. Human iPSC-CMs were treated with remdesivir in the presence/absence of mdivi-1 (MD, 15 mM) (B) Mitochondrial morphology was assessed using mitotracker staining, n=6. MD co-treatment with 2.5 mM remdesivir increased the proportion of cells with elongated mitochondria (asterisk), while cells treated with remdesivir alone displayed mostly punctate (arrowhead) and perinuclear (arrow) mitochondria. (C) Mitochondrial redox activity was measured using the PrestoBlue assay, n=7. (D) Confocal images of hiPSC-CMs showing MLC2V-eGFP signal in green, Hoescht nuclear staining in blue. Sparse and truncated sarcomeres (arrowheads), patchy MLC2V-eGFP signal with no or poorly discernible organisation (arrows) were enriched in cells with remdesivir alone, while MD co-treatment resulted to more striated sarcomeres. Statistical significance was calculated relative to control cells using the one-way ANOVA with Dunnett's relative to control cells for (A) and with Sidak's multiple comparisons test against cells treated with remdesivir alone without MD for (C) respectively, and two-way ANOVA with Sidak's multiple comparisons test against cells treated with remdesivir alone without MD for (B). *p<0.05, **p<0.01, ***p<0.001, ****p<0.0001.

FIG. 6. Characterization of hiPSC-CMs. (A) Schematic of cardiac differentiation at different days (D) of differentiation. (B) Flow cytometric analysis of hiPSC-CMs. D20 hiPSC-CMs were stained with an antibody against troponin T (cTnT, blue) and isotype control (red). (C) Human iPSC-CMs were stained with anti-α-actin antibody. Fluorescent images of MLC2V-eGFP signal, α-actin staining, and DAP1 nuclear staining are shown in green, red, and blue, respectively. Small images below show magnified views of MLC2V-eGFP and α-actin striations, with clear intercalated patterns indicative of localization at A-bands and Z-disks, respectively.

FIG. 7. Analysis of nuclear morphology in hiPSC-CMs. Human iPSC-CMs were treated with indicated concentrations of remdesivir under normoxic and hypoxic (2.5% 02) conditions for 3 days, stained with the Hoescht nuclear dye and imaged with a fluorescent microscope, n=3.

FIG. 8. DRP1 and mitochondrial fragmentation as a general mediator/features of NA-induced toxicity. (A) Manual docking against DRP1 correlates with known cardiotoxicity. The triphosphate metabolite of NAs were manually docked into the GTPase domain of DRP1. A negative score predicts high affinity binding. Compounds previously associated with cardiotoxicity (red) had high affinity, while those with minimal or no known cardiotoxicity (black) had low affinity. (B) Mitochondrial morphology was examined using the MTDR dye. Control CMs and those treated with ribavirin (generally considered safe) primarily had elongated mitochondria, while CMs treated with BMS-986094 (lethal cardiotoxicity) and another anti-COVID-19 NA (Molnupiravir metabolite β-D-N4-hydroxycytidine) had punctate (arrowhead) or perinuclear (arrow) mitochondria. (C) Remdesivir, BMS-986094 and Molnupiravir (Molnupiravir metabolite β-D-N4-hydroxycytidine) reduced the proportion of cells with healthy elongated mitochondria, while ribavirin did not have noticeable effect.

### DEFINITIONS

The terms “a,” “an,” and “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “about” and “approximately” as used herein shall generally mean an acceptable degree of error for the quantity measured given the nature or precision of the measurements. “About X” denotes a range of +/−10% of a pre-determined value X: for example, “about 10” defines a range of 10±1 or 9−11.

“DRP1” or “dynamin-related protein” is to an 80-kDa mechanochemical GTPase of the dynamin superfamily. It mediates mitochondrial and peroxisomal fission (division) in mammals during a process dependent on self-assembly and coupled to GTP hydrolysis. The DRP1 protein shuttles between the cytosol and these organelles, where it is recruited to potential fission sites. DRP1 then polymerizes at some of these foci, leading to membrane scission. The amino acid sequence for human DRP1 protein is known, for example, set forth in UniProtKB-000429 and provided herein as SEQ ID NO:1, with its GTPase domain located in the segment 1-301 of SEQ ID NO:1.

The term “toxicity” or “cytotoxicity” is used to describe the quality of a substance in causing detectable loss of cellular functions up to and including cell death. A cytotoxic compound can cause tissue and organ damage due to damage or death at a cellular level. Cell death caused by cytotoxicity maybe through either necrosis or apoptosis. A cell-based cytotoxicity assay is an experimental system useful in this context for detecting a compound's potential cytotoxicity by assessing the compound's effects on cells (especially a specific cell type of interest, such as hiPSC-CMs) that have been exposed to the compound in an effective amount or concentration. Cytotoxicity as manifested in morphological, structural, and functional anomalies at cellular, intracellular, and molecular levels may be detected and quantitatively measured by a number of established methodologies including, but not limited to, counting of live/viable cells to determine cell death/survival rate and/or percentage viability, monitoring mitochondrial, nucleic, structural, and electrophysiological abnormalities in cells, e.g., measurement of mitochondrial redox activity, analysis of morphology changes in cellular components (mitochondria, nuclei etc.) indicative of dysfunction (such as fission or fragmentation, nuclear condensation, and perturbations in sarcomeric arrangement), measurement of mitochondrial superoxide level, and measurement of mitochondrial membrane potential (Δψm). Typically, a cell-based cytotoxicity assay includes both positive and negative controls, i.e., compounds that are known to possess or not possess a detectable toxic effect in the particular assay format, to ensure the proper detection and measurement of cytotoxicity.

The term “molecular docking,” or simply “docking,” when used in the context of molecular modeling, refers to a method by which one predicts the preferred spatial orientation of one molecule (especially a macromolecule such as a protein, peptide, nucleic acid, carbohydrate, or lipid) in relation to another molecule when they are bound to each other to form a stable receptor-ligand complex. Such knowledge of the preferred orientation in turn may be used to predict the strength of association or binding affinity between two molecules using, for example, scoring functions. The ability of a macromolecule such as a protein to interact with a small molecule ligand to form a supramolecular complex plays a major role in the dynamics of the protein, which may enhance or inhibit its biological function. The behavior of a small molecule in the binding pocket of a target protein can be described by molecular docking, which aims to identify correct or preferred poses of a ligand in the binding pocket and to predict the affinity between the ligand and the protein. Based on the types of ligand, docking can be classified as protein—small molecule (ligand) docking, protein—nucleic acid docking, and protein—protein docking. Many computer-simulated programs have been developed to perform molecular docking for a variety of purposes including structure-based new drug discovery and development. See, e.g., “Use of molecular docking computational tools in drug discovery,” Francesca Stanzione et al., in Progress in Medicinal Chemistry, 2021.

The term “immunoassay” describes an assay that detects qualitatively and/or quantitatively the presence of a molecule based on the specific binding relationship between an antibody and its antigen. The immunoassay is characterized by the use of specific binding properties of a particular antibody to identify, isolate, target, and/or measure the antigen. The phrase “specifically binds,” when used to describe the binding relationship between an antibody and its target antigen such as in the context of an immunoassay, refers to a binding reaction that is determinative of the presence of the antigen (e.g., a polypeptide such as the DRP1 protein) in a heterogeneous population of other biologics. Thus, under designated immunoassay conditions, the specified antibodies bind to a particular polypeptide at least two times the background and do not substantially bind in a significant amount to other polypeptides or other antigens present in the sample. Specific binding to an antibody under such conditions may require an antibody that is selected for its specificity for a particular protein. For example, polyclonal antibodies raised to a DRP1 protein having the amino acid sequence of SEQ ID NO:1, can be selected to obtain only those polyclonal antibodies that are specifically immunoreactive with that specific protein and not with other proteins. This selection may be achieved by subtracting out antibodies that cross-react with other unrelated antigens. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically, a specific binding reaction will yield at least twice of the background signal and more typically more than 5, 10, 20, 50, or up to 100 times the background.

As used herein, the term “binding affinity” describes the strength of the binding interaction between a single biomolecule (e.g., a protein such as the DRP1 protein) to its ligand/binding partner (e.g., a proposed therapeutic agent). Binding affinity is typically measured and reported by the equilibrium dissociation constant (K_{D}), which is used to evaluate and rank order strengths of bimolecular interactions. The smaller the K_{D} value, the greater the binding affinity of the ligand for its target. The larger the K_{D} value, the more weakly the target molecule and ligand are attracted to and bind to one another. Typically, a binding affinity of no greater than 10⁻⁵ M or 10⁻⁶ NI indicates presence of a specific binding relationship between a biomolecule (e.g., the DRP1 protein) and a second compound. In addition, the presence of a specific binding relationship between a biomolecule and a second compound can also be indicated by a binding signal that is at least twice, 5 times, 10 times, 20 times, 30 times, 50 times, 100 times or even higher than the background binding signal between the biomolecule and a compound that is known as a confirmed non-binder of the biomolecule.

The term “increase” or any of its grammatical variations, as used herein, refers to any detectable positive effect on a target biological process, such as the binding between a compound and the DRP1 protein, by at least 50%, 100%, 2 fold, 5 fold, 10 fold, 20 fold or more, when compared to a control level such as a negative control level. Similarly, the term “decrease” or its variations refers to any detectable negative effect on a target biological process, such as the binding between a compound and the DRP1 protein, by a reduction of at least 10%, 20%, 30%, 40%, 50%, 60%, 70%, 80% or higher in DRP1 binding when compared to a control level such as a positive control level indicated by the binding signal generated from DRP1 protein with a known binder.

### DETAILED DESCRIPTION

### I. Introduction

During their study of remdesivir-induced cardiotoxicity, the present inventors discovered that the cytotoxicity can be ameliorated by inhibition of mitochondrial fission. The mRNA level of DRP1, a master regulator of mitochondrial fission, was revealed to elevate substantially and in a dose-dependent manner in the cells in response to exposure to remdesivir. Moreover, known cytotoxic agents, including compounds within the category of nucleotide/nucleoside analogues, which are often proposed for use in treating infectious diseases, especially viral infections, have been shown to induce mitochondrial fission, which is a sign of high DRP1 activity. It is based on this discovery that the present inventors have now devised rapid screening methods for identifying compounds among proposed therapeutics as potentially presenting toxicity to recipients based on their specific interaction with the DRP1 protein and therefore requiring further investigation in this regard.

### II. Production and Detection of DRP1 Protein

A. General Recombinant Technology

Basic texts disclosing general methods and techniques in the field of recombinant genetics include Sambrook and Russell, Molecular Cloning, A Laboratory Manual (3rd ed. 2001); Kriegler, Gene Transfer and Expression: A Laboratory Manual (1990); and Ausubel et al., eds., Current Protocols in Molecular Biology (1994).

For nucleic acids, sizes are given in either kilobases (kb) or base pairs (bp). These are estimates derived from agarose or acrylamide gel electrophoresis, from sequenced nucleic acids, or from published DNA sequences. For proteins, sizes are given in kilodaltons (kDa) or amino acid residue numbers. Proteins sizes are estimated from gel electrophoresis, from sequenced proteins, from derived amino acid sequences, or from published protein sequences.

Oligonucleotides that are not commercially available can be chemically synthesized, e.g., according to the solid phase phosphoramidite triester method first described by Beaucage & Caruthers, Tetrahedron Lett. 22: 1859-1862 (1981), using an automated synthesizer, as described in Van Devanter et. al., Nucleic Acids Res. 12: 6159-6168 (1984). Purification of oligonucleotides is performed using any art-recognized strategy, e.g., native acrylamide gel electrophoresis or anion-exchange HPLC as described in Pearson & Reanier, J. Chrom. 255: 137-149 (1983).

The sequence of a human DRP1 gene, a polynucleotide encoding a polypeptide having the amino acid sequence SEQ ID NO:1, and synthetic oligonucleotides can be verified after cloning or subcloning using, e.g., the chain termination method for sequencing double-stranded templates of Wallace et al., Gene 16: 21-26 (1981).

B. Chemical Synthesis of DRP1 Protein

The amino acid sequence of human DRP1 protein and the boundaries of its GTPase domain are provided. A polypeptide comprising the full-length DRP1 protein or a segment thereof including the GTPase domain thus can be chemically synthesized using conventional peptide synthesis or other protocols well-known in the art.

Polypeptides may be synthesized by solid-phase peptide synthesis methods using procedures similar to those described by Merrifield et al., J. Am. Chem. Soc., 85:2149-2156 (1963); Barany and Merrifield, Solid-Phase Peptide Synthesis, in The Peptides: Analysis, Synthesis, Biology Gross and Meienhofer (eds.), Academic Press, N.Y., vol. 2, pp. 3-284 (1980); and Stewart et al., Solid Phase Peptide Synthesis 2nd ed., Pierce Chem. Co., Rockford, Ill. (1984). During synthesis, N-α-protected amino acids having protected side chains are added stepwise to a growing polypeptide chain linked by its C-terminal and to a solid support, i.e., polystyrene beads. The peptides are synthesized by linking an amino group of an N-α-deprotected amino acid to an α-carboxy group of an N-α-protected amino acid that has been activated by reacting it with a reagent such as dicyclohexylcarbodiimide. The attachment of a free amino group to the activated carboxyl leads to peptide bond formation. The most commonly used N-α-protecting groups include Boc, which is acid labile, and Fmoc, which is base labile.

Materials suitable for use as the solid support are well known to those of skill in the art and include, but are not limited to, the following: halomethyl resins, such as chloromethyl resin or bromomethyl resin; hydroxymethyl resins; phenol resins, such as 4-(α-[2,4-dimethoxyphenyl]-Fmoc-aminomethyl)phenoxy resin; tert-alkyloxycarbonyl-hydrazidated resins, and the like. Such resins are commercially available and their methods of preparation are known by those of ordinary skill in the art. Briefly, the C-terminal N-α-protected amino acid is first attached to the solid support. The N-α-protecting group is then removed. The deprotected α-amino group is coupled to the activated α-carboxylate group of the next N-α-protected amino acid. The process is repeated until the desired peptide is synthesized. The resulting peptides are then cleaved from the insoluble polymer support and the amino acid side chains deprotected. Longer peptides can be derived by condensation of protected peptide fragments. Details of appropriate chemistries, resins, protecting groups, protected amino acids and reagents are well known in the art and so are not discussed in detail herein (See, Atherton et al., Solid Phase Peptide Synthesis: A Practical Approach, IRL Press (1989), and Bodanszky, Peptide Chemistry, A Practical Textbook, 2nd Ed., Springer-Verlag (1993)).

C. Recombinant Expression of DRP1 Protein

A DRP1 protein or fusion polypeptide comprising at least one fragment of the DRP1 protein and its GTPase domain can be produced using routine techniques in the field of recombinant genetics, relying on the polynucleotide sequences encoding the polypeptide disclosed herein.

To obtain high level expression of a nucleic acid encoding a desired polypeptide, one typically subclones a polynucleotide encoding the polypeptide into an expression vector that contains a strong promoter to direct transcription, a transcription/translation terminator and a ribosome binding site for translational initiation. Suitable bacterial promoters are well known in the art and described, e.g., in Sambrook and Russell, supra, and Ausubel et al., supra. Bacterial expression systems for expressing the polypeptide are available in, e.g., E. coli, Bacillus sp., Salmonella, and Caulobacter. Kits for such expression systems are commercially available. Eukaryotic expression systems for mammalian cells, yeast, and insect cells are well known in the art and are also commercially available. One exemplary eukaryotic expression vector is an adenoviral vector, an adeno-associated vector, or a retroviral vector.

Standard transfection methods can be used to produce bacterial, mammalian, yeast, insect, or plant cell lines that express large quantities of a recombinant polypeptide (e.g., DRP1 protein), which is then purified using standard techniques (see, e.g., Colley et al., J Biol. Chem. 264: 17619-17622 (1989); Guide to Protein Purification, in Methods in Enzymology, vol. 182 (Deutscher, ed., 1990)). Transformation of eukaryotic and prokaryotic cells are performed according to standard techniques (see, e.g., Morrison, J Bact. 132: 349-351 (1977); Clark-Curtiss & Curtiss, Methods in Enzymology 101: 347-362 (Wu et al., eds, 1983).

Any of the well-known procedures for introducing foreign nucleotide sequences into host cells may be used. These include the use of calcium phosphate transfection, polybrene, protoplast fusion, electroporation, liposomes, microinjection, plasma vectors, viral vectors and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell capable of expressing the recombinant polypeptide.

When a recombinant polypeptide, e.g., a DRP1 protein, is expressed in host cells in satisfying quantity, its purification can follow the standard protein purification procedure including Solubility fractionation, size differential filtration, and column chromatography. These standard purification procedures are also suitable for purifying DRP1 polypeptides obtained from chemical synthesis. The identity of the DRP1 protein may be further verified by methods such as immunoassays (e.g., Western blot or ELISA) and mass spectrometry.

### III. Assessing Binding of a Test Compound to DRP1 Protein

A. Molecular Docking

One method for rapidly assessing the binding potential between a test compound, e.g., a compound that has been proposed for use as a therapeutic agent in medical applications, and the DRP1 protein, as the first indication of possible cytotoxicity of the compound relies on the tool known as molecular docking.

A computer-based methodology, molecular docking was initially designed to predict the binding of small drug-like molecules to target proteins. As many diseases are caused by the malfunction of proteins and therapies are focused on the inhibition or activation of the target proteins, traditional lead generation methods for drug discovery normally entail assaying a large variety of interesting compounds against a specific protein known to be a disease target and hoping to observe a binding interaction. While more protein structures are determined experimentally using X-ray crystallography or nuclear magnetic resonance (NMR) spectroscopy, molecular docking is increasingly used as a tool in drug discovery. In the context of the present invention, molecular docking can be used to virtually screen new compounds in a similar way to experimental high-throughput screening as well as offering atomistic level insight to facilitate structure-based assessment of a binding relationship between the DRP1 protein and a test compound as a rapid and effective means for preliminarily identifying compounds of possible cytotoxicity, which, if preferred, may be further tested to confirm or eliminate the suspected cytotoxicity.

The technology involved in molecular docking methodology has been in keen development for the past decades. A variety of software programs are now readily available for use in exploration of intermolecular interaction and are suitable either directly or upon modification in the practice of the present invention. Upon input of the specific chemical composition of a potential binding pair, such as the DRP1 protein and a compound being analyzed for its potential cytotoxicity, such software programs can predict a 3-dimensional binding orientation for the pair and generate a score corresponding to the predicted binding affinity. This score may then be utilized to assess the propensity of the two molecules, e.g., the test compound and the DRP1 protein, as a binding pair. For the purpose of the present invention, a pre-selected threshold value (e.g., a pre-determined value generated by the same software based on the binding between the DRP1 protein and a cytotoxic compound known to bind the DRP1 protein) may be used to indicate a positive finding in the binding assessment (e.g., between the DRP1 protein and another compound of unknown cytotoxicity profile). For a review of molecular docking software, see, e.g., Pagadala et al., Biophys Rev. 2017 April; 9(2): 91-102.

B. Physical Binding Assays

A second approach in assessing the potential cytotoxicity of a compound focuses on the physical interaction between the compound and the DRP1 protein. As observed in this study, compounds that modulate the activity of the DRP1 protein may exert such effects by directly interacting with the protein, especially through its GTPase domain. As such, an in vitro or cell-free screening method effective for providing a preliminary indication of whether a molecule is a DRP1 modulator relied on the detection of physical interaction or specific binding between a test compound and the DRP1 protein (especially at the GTPase domain).

Typically, a compound being screened for cytotoxicity is first placed together with the DRP1 protein, or a segment of the DRP1 protein containing the GTPase domain, or a fusion protein encompassing such a segment of DRP1 protein, under conditions generally allowing any potential binding between the compound and the DRP1 protein (e.g., in an aqueous solution with appropriate salts and pH), the physical association between the test compound and the DRP1 protein is then detected and quantitatively measured, for example, by determining a Kd value. If an increased level of association is observed in comparison with the association level between the same version of DRP1 protein and a “negative control” compound known to not specifically physically interact/bind with the DRP1 protein, for example, by comparing the two Kd values, the test compound is preliminarily deemed a compound with likely cytotoxicity. Conversely, if no increase in the level of association is observed compared to the “negative control” association level (e.g., by comparing the two Kd values), the test compound is preliminarily deemed as likely without cytotoxicity.

Optionally, a compound that is preliminarily identified as possibly toxic may be subject to further testing and investigation, for example, in cell-based assays, by detecting its potential cytotoxic effects on one or more types of cells (e.g., cardiomyocytes) as manifested in cell death, loss of viability or functions, etc. Similarly, compounds preliminarily identified as probably non-toxic can also be further tested and verified in various cell-based assays or even animal models as truly possessing no detectable cytotoxicity.

The initial screening step carried out in an in vitro or cell-free setting is suitable to be adapted in a high throughput system for simultaneously screening for a large number of test compounds for their potential binding to the DRP1 protein. For example, an array of multiple test compounds having been immobilized to a solid substrate or support with each compound located at a distinct, pre-assigned, and thus individually identifiable location on the array may be contacted with the DRP1 protein under conditions permissible for the compounds to bind to the DRP1 protein. The presence of specific binding, defined as at least twice, preferably at least 5 time, 10, 20, 50, or 100 times over the background or “negative control” binding signal, between a test compound and the DRP1 protein is then detected based on the presence of the DRP1 protein (e.g., as determined by an immunoassay) at an individually identifiable location on the array.

### IV. Further Testing for Cytotoxicity

The use of molecular docking or physical binding assays can provide a quick and preliminary indication of a compound of interest as potentially possessing cytotoxicity. This suspected toxic compound, if desired, can be further investigated to first confirm this suspected cytotoxicity and then determine the level and nature of such cytotoxicity. On the other hand, a compound not found to possess cytotoxicity during the initial screening step may also be subject to further testing so that it can be confirmed as without significant cytotoxicity and therefore presents as a promising candidate for future drug development.

As a compound's cytotoxicity may manifest itself in the ability to cause cell death (necrosis or apoptosis) and/or loss of cellular functions, a variety of suitable cell-based assays are useful for the second stage of the present invention for the purpose of further testing, confirmation or elimination of potential cytotoxicity.

Mitochondria are double-membrane-bound organelles found in the cytosol of most eukaryotic cells. Functioning as the “powerhouse of the cell,” mitochondria generate most of a cell's supply of adenosine triphosphate (ATP), which is subsequently used throughout the cell as a source of chemical energy. Because of their critical importance in cell viability and functionality, morphological, structural, or chemical changes in mitochondrial are useful and reliable indicators of cellular damages caused by exposure to cytotoxic agents. Thus, assays that monitor changes in mitochondria morphology (e.g., detecting increased fragmentation or fission of mitochondria as well as other morphological anomaly), mitochondria redox activity, mitochondria superoxide level, and mitochondria membrane potentials can be employed in the second stage of testing to confirm or eliminate any suspected cytotoxicity caused by a test compound.

Damages to cells due to cytotoxic effects from a test compound can also be studied during the second stage testing of the present invention. In this regard, one or more types of cells maintained in culture may be monitored for any changes in their functionality upon exposure to a test compound. For example, cardiomyocytes may be monitored for any changes in their electrophysiological properties following incubation in the presence of a test compound. Anomalies in their electrophysiological profiles resulted from exposure to a test compound indicate the compound's cytotoxicity to this cell type and to the cardiovascular system. In some but not all cases, cytotoxicity to cells will result in cell death and therefore can be detected in studies monitoring changes in the proliferation and/or survival rate of cells following exposure to the compound. A variety of assays measuring live cells, including flow cytometry-based assays, are available for this purpose.

Lastly, cytotoxicity of a test compound may be detected and assessed in suitable animal models. For example, cardiac toxicity of a compound can be monitored in a mammal (e.g., a rodent) upon systemic injection (e.g., intravenous, intramuscular, or subcutaneous injection) of the compound to the animal. Cardiomyopathy, myocarditis/pericarditis, acute coronary syndromes, and congestive heart failure manifested in symptoms such as abnormal heart rhythm (arrhythmia), difficulty breathing, physical exhaustion/extreme fatigue without significant exertion, etc. are indicators of cardiac toxicity.

### EXAMPLES

The following examples are provided by way of illustration only and not by way of limitation. Those of skill in the art will readily recognize a variety of non-critical parameters that could be changed or modified to yield essentially the same or similar results.

### INTRODUCTION

Coronavirus disease of 2019 (COVID-19), which is caused by severe acute respiratory syndrome coronavirus 2 (SARS-CoV-2), has claimed millions of casualties worldwide. Although COVID-19 primarily affects the respiratory system, cardiac damage is prevalent, occurring in 20-30% of hospitalised patients and contributing to 40% of deaths.¹ The urgency of the COVID-19 pandemic has demanded the rapid development of therapeutic strategies. To this end, remdesivir, a compound originally designed for the treatment of Ebola virus disease, has been repurposed for use against COVID-19. Remdesivir has broad spectrum activity against multiple RNA viruses and inhibits SARS-CoV, MERS-CoV, and SARS-CoV-2 in vitro and in vivo.²⁻⁴ In a double-blind, placebo-controlled multicentre clinical trial, 1062 persons with severe COVID-19 were randomised to receive a 10-day treatment of remdesivir or placebo.⁵ Patients who received remdesivir experienced a statistically significant decline in the median recovery time to 10 days vs 15 days in the placebo group. Mortality was reduced to 6.7% (remdesivir) vs 11.9% (placebo) by day 15, and 11.4% (remdesivir) vs 15.2% (placebo) by day 29.⁵ These results are consistent with a smaller placebo-controlled trial involving 237 patients which showed a statistically non-significant faster time to clinical improvement in patients who received remdesivir compared to control (21 days vs 23 days), particularly in those who received the drug within 10 days of onset of symptoms (16 vs 23 days)⁶. These results are contradictory to the larger open-label WHO Solidarity trial, in which no clinical or survival benefit was observed.⁷ Among patient subgroups, those with moderately severe disease (requiring supplemental oxygen) responded better than those with mild (not requiring oxygen) and severe disease (requiring high flow oxygen or mechanical ventilation).⁵ Serious adverse events were detected at similar rates in the remdesivir treatment vs placebo group.⁵ Remdesivir was first granted emergency use authorisation for the treatment of COVID-19 patients on the May 1, 2020, and was the first and, at the time of this study, the only drug to receive formal approval from the US Food and Drug Administration (FDA) on the Oct. 22, 2020, for use in adults and children hospitalised with suspected or laboratory confirmed COVID-19.⁸

Remdesivir is a prodrug of an adenosine triphosphate analogue which binds to RNA-dependent RNA polymerase to inhibit viral replication.⁹ Nucleotide/nucleoside analogues are important treatment against RNA viruses but their use have been associated with increased incidences of mitochondrial toxicity, which mainly affects tissues with high energy demand such as the heart.¹⁰ The mitochondria comprise 30% of the volume of adult cardiomyocytes (CMs), and are critical for cardiac metabolism and apoptosis. Damage to this organelle can therefore severely impair cardiac function. For instance, nucleotide/nucleoside analogues such as azidothymidine and abacavir have been associated with increased risk of cardiovascular disease.¹¹ Toxicity is largely attributed to inhibition of Pol-γ¹² and POLRMT, the DNA and RNA polymerases responsible for the synthesis and transcription of mitochondria DNA. Other mechanisms of mitochondrial interference, such as direct inhibition of and alterations in the expression of mitochondrial genes and proteins¹⁴, enhanced production of mitochondrial reactive oxygen species' have been proposed. Although there have been few reports of adverse cardiac effects related to the use of remdesivir, clinical data are currently limited.

Patients with COVID-19 are vulnerable to treatment induced cardiotoxicity due to the high prevalence of cardiovascular damage among these individuals.¹⁶ Many factors may contribute to heart injury including pre-existing cardiovascular co-morbidities^{17, 18}, direct viral infection of CMs¹⁹⁻²³, acute inflammation and myocarditis²⁴, and hypothermia-induced cardiac damage²⁵. Acute inflammation in the lungs can compromise respiratory function, leading to low oxygen saturation in the blood and hypoxia in the heart. Dypsnea and low oxygen saturation are observed in 50-60% of hospitalised patients with COVID-19, and are associated with greater risk of cardiac damage and poor prognosis.^{26, 27} The heart is metabolically demanding, and is reliant on oxygen to drive oxidative phosphorylation in the mitochondria. Increased cardiometabolic demand associated with systemic infection, coupled with hypoxia caused by acute respiratory illness can disturb the balance between myocardial oxygen demand and supply to result in mitochondrial dysfunction and injury. Remdesivir was initially indicated for use in patients with moderate/severe COVID-19 who requires supplemental oxygen, although this was later broadened to all hospitalised patients. In moderate/severe patients, systemic hypothermia may damage the heart, and thereby increases susceptibility to the potential adverse effects of remdesivir.

Human induced pluripotent stem cells (hiPSCs) can self-renew in culture; their differentiation to the cardiac lineage represents a potentially unlimited source of CMs for disease modelling and cardiotoxicity testing.^{28-30.} Human iPSC derived CMs (hiPSC-CMs) spontaneously contract, express genes/proteins associated with cardiac identity and recapitulate key aspects of human cardiac physiology^{28, 31-34}. Specifically, hiPSC-CMs have been shown to respond to agents which damage the mitochondria and are now an important component of cardiotoxicity testing.^{29, 30, 34} In the context of COVID-19, hiPSC-CMs were recently used to demonstrate direct infection of CMs by SARS-CoV-2.^{19, 20, 23, 35, 36} Sarcomeric disarray, cessation of beating, electrical and contractile disturbances, and apoptosis were observed after infection.^{19, 36} Transcriptional analysis further revealed significant downregulation of genes important for mitochondrial function and oxidative phosphorylation in infected hPSC-CMs.¹⁹ Human iPSC-CMs are therefore a suitable platform for the evaluation of the adverse cardiac effects of COVID-19 treatment.

In this study, the present inventors investigated the cardiotoxic effects of remdesivir by exposing hiPSC-CMs to clinically relevant concentrations of this drug under normoxic and hypoxic conditions to simulate (i) prophylactic use in healthy individuals and treatment for patients with mild COVID-19, and (ii) therapeutic use in patients with severe COVID-19 suffering from pneumonia induced hypoxemia respectively. Remdesivir induced mitochondrial dysfunction in the form of reduced redox potential and mitochondrial fragmentation, and structural abnormalities at concentrations several folds below Cmax under both normoxic and hypoxic conditions. Importantly, these changes persisted and worsened after the withdrawal of this drug. Inhibition of mitochondrial fission ameliorated remdesivir induced damage, showing that disturbed mitochondrial dynamics was a mechanistic contributor to the cardiotoxic effects of remdesivir.

### Methods

Human iPSC Culture and Cardiac Differentiation

The hiPSC line AICS-0060-027 (Allen Cell Institute) was used for experiments unless otherwise indicated. The cell line is a derivative of the parental line (WTC-11) and contains a mono-allelic mEGFP-tagged MYL2 modification. The genetic engineering, generation/selection of clones were performed by Allen Cell Institute. Undifferentiated hiPSCs (AICS-0060-027) were maintained on Matrigel (Corning Life Science, Tewksbury, Mass.)-coated surfaces at 37° C. and 5% CO₂ in mTeSR medium plus supplements (Stem Cell Technologies, Vancouver, BC, Canada). Differentiations of hiPSC to CMs was performed as per supplier's instructions with slight modifications. Three days prior to differentiation, hiPSCs were plated into individual wells of a 6-well plate in mTeSR medium. To initiate differentiation, the mTeSR medium was changed to RPMI/B27 medium lacking insulin (Thermo Fisher Scientific, Waltham, Mass.). CHIR99021 (7.5 μM; Cayman Chemical, Ann Arbor, Mich.) was added to the cells from days 0 to 2, followed by the addition of IWR-1-endo (5 μM; Cayman Chemical) and IWP-2 (7.5 μM; APExBIO, Boston, Mass.) from days 2 to 4. On day 8, cultures were maintained in RPMI/B27 medium containing insulin (Thermo Fisher Scientific) and medium was changed at 3-day intervals, except on day 18-21, when cultures were kept in RPMI (no glucose)/B27 supplemented with 1× Linoleic Acid-Albumin, 1× Oleic Acid-Albumin and T₃ (10 nM; Sigma-Aldrich, St. Louis, Mo.) for metabolic selection of CMs. Human iPSC-CMs were used for experiments after day 40.

### Flow Cytometry Measurements

Differentiated CM cultures were analysed by flow cytometry using antibodies to TNNT2. Cells were rinsed with DMEM/F12 and dissociated with 0.05% trypsin/EDTA for 5-10 min (Thermo Fisher Scientific) at 37° C., and neutralised with DMEM/12 with 10% FBS. Cells were washed 2× with wash buffer (1% FBS in Dulbecco's PBS (DPBS) (no Mg²⁺, no Ca²⁺) pH 7.4) and fixed with 4% paraformaldehyde for 15 min at RT, followed by methanol for 15 min at 4° C. Cells were then washed once with 0.5% BSA in DPBS. The cells were then incubated with the primary antibody at a dilution of 1:200 for 1 h, after which they were washed once with 0.5% BSA/0.1% Triton X-100 in DPBS, incubated with the anti-IgG1 secondary antibody with 1:1000 dilution for 30 min, followed by one more wash. Isotype controls were used at the same concentration as the primary antibody. Immunostained cells were resuspended in 0.5% BSA in DPBS and analysed on a CytoFLEX flow cytometer (Beckman Coulter). A minimum of 10,000 events were acquired and data were analysed with the FlowJo software. The percentage of positive cells was based on gated cells with a background contribution of <2%.

### Immunofluorescence Staining

Cultures of differentiated hiPSC-CMs were dissociated with 0.05% Typsin/EDTA and plated onto matrigel-coated glass plate in RPMI/B27 medium containing insulin (Thermo Fisher Scientific). Samples were fixed with PBS containing 4% paraformaldehyde for 15 min at room temperature, and then permeabilised in PBS containing 1% Triton X-100 at room temperature for 15 min. After blocking with 10% FBS in PBS at room temperature for 1 h, samples were incubated with anti-α-actinin antibody (Abcam, Cambridge, Mass.) in PBS containing 10% FBS at 4° C. overnight. After washing, secondary antibodies (Thermo Fisher Scientific) were applied for 1 h at room temperature in the dark. The cells were mounted with ibidi Mounting Medium (ibidi USA Inc., Fitchburg, Wis.). The samples were imaged with a Leica THUNDER Imager microscope.

### Measurements of Mitochondrial Redox Activity

Mitochondrial redox activity was measured using the resazurin-based PrestoBlue assay (Thermo Fisher Scientific), according to the manufacturer's instructions by the addition of Presto blue reagent to CMs for 30 mins at 37° C. The intensity of fluorescence was measured using the Synergy HTX plate-reader (Biotek, Winooski, Vt.) with excitation and emission wavelengths of 540 nm and 590 nm, respectively. Control=0.0125% DMSO, to match the DMSO content of the highest dose of remdesivir used, and was not significantly different from untreated control. Data was normalised to that of untreated cells.

### Evaluation of Mitochondrial Morphology

Cells were incubated with Mitotracker Deep Red FM dye (200 nM; Thermo Fisher Scientific) and Hoescht 33342 dye (5 μg/ml; Thermo Fisher Scientific) for 30 min at 37° C., after which they were fixed with 4% paraformaldehyde at room temperature for 15 min followed by 15 min wash with PBS and imaged with a Leica THUNDER Imager microscope or Nikon AIR HD25 confocal microscope.

Mitochondria was scored as elongated, punctate and perinuclear by a technician blinded to the identities of the samples. Classification is as follows:
Elongated mitochondria: continuous and filamentous pattern throughout the cell.
Punctate mitochondria: punctate staining throughout the cell.
Perinuclear mitochondria: intense staining around the nucleus, with little staining through the cells.

Similarly, nuclei were scored as normal (relatively large nuclei with regular shape) or condensed (intensely stained nuclei with irregular shape) by a technician blinded to the identities of the samples.

### Mitochondrial Superoxide Measurements

Mitochondrial superoxide (O₂⁻) levels were assayed using the MitoSOX™ Red reagent (Thermo Fisher Scientific), according to the manufacturer's instructions. CMs were treated with 2.5 μM MitoSOX™ Red reagent for 10 min at 37° C. After washing, samples were imaged with a Leica THUNDER Imager microscope, using excitation and emission wavelengths of 470 nm and 590 nm. Fluorescence intensities were quantified using ImageJ.

### Mitochondrial Membrane Potential (Δψm) Measurements

Δψm was measured using the tetramethylrhodamine, ethyl ester (TMRE), a potentiometric dye whose fluorescent intensity is proportional to the mitochondrial potential. CMs were plated into Matrigel-coated surfaces in RPMI/B27 medium. Cells were incubated with TMRE (25 nM; Thermo Fisher Scientific) for 30 min at 37° C., after which they were washed with DPBS and intensity of fluorescence was measured using the Synergy HTX plate-reader (Biotek, Winooski, Vt.) with excitation and emission wavelengths of 540 nm and 590 nm.

Quantitative Real Time PCR (qRT-PCR) Measurements

Total RNA was extracted using Trizol Reagent according to the manufacturer's instructions (Thermo Fisher Scientific). Extracted RNA was reverse transcribed using PrimeScript RT Master Mix (Takara). Quantitative RT-PCR of target genes was performed with GoTaq® qPCR Master Mix (Promega) using an QuantStudio™ 12K Flex. Gene transcript abundance was normalised to B2M and then to that of the untreated sample to control for biological variations among the different sets of samples. Primer sequences are shown in Table 1.

### Statistical Analysis

One-way ANOVA with Dunnett's multiple comparisons test was employed to determine statistical significance between all experimental groups except for assessment of mitochondrial morphology. For evaluations of mitochondrial morphology, 2-way ANOVA analysis was conducted with matching factors, with Dunnett's multiple comparisons test, to evaluate the incidence of different mitochondrial morphologies in response to different doses of remdesivir. For pairwise comparison of mitochondrial morphology in the presence/absence of mdivi-1, 2-way ANOVA analysis was conducted with matching factors, with Sidak's multiple comparisons test. Analyses were done using the GraphPad Prism software. Results are presented as mean±SEM (standard error of the mean), with n values indicated in the Figure Legend. The asterisks indicate data that are significantly different at *p<0.05, **p<0.01, ***p<0.001, ****p<0.0001.

### Results

Remdesivir Induced Mitochondrial Abnormalities in hiPSC-CMs

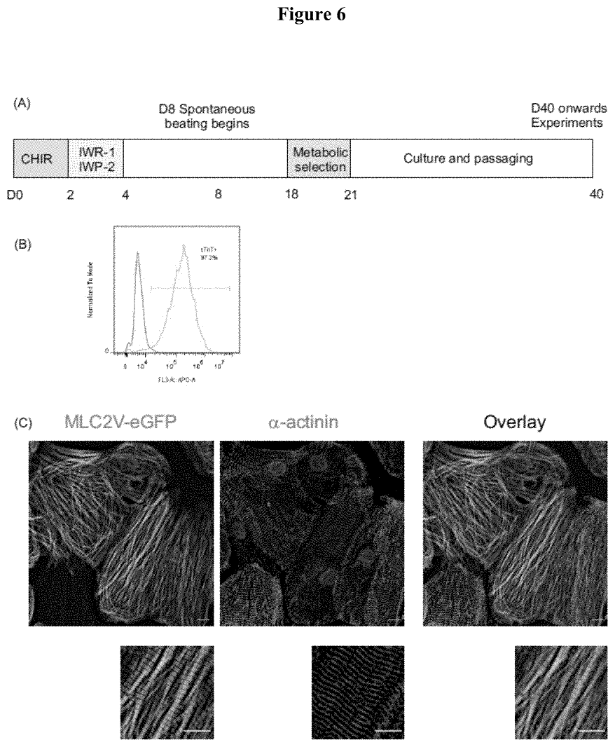

An hiPSC line (AICS-0060-027) was utilised in which the cardiac ventricular marker, MLC2V, is tagged with the eGFP fluorescent reporter. Human iPSC-CMs were generated using an established monolayer differentiation protocol via modulation of the WNT signalling pathway, followed by metabolic selection (FIG. 6A)³⁷. Spontaneous contractions could be observed by days 7-8 of differentiation. Flow cytometry experiments showed that hiPSC cardiac cultures were >95% positive for cardiac Troponin-T, an established marker of CMs (FIG. 6B), and MLC2V-eGFP⁺ ventricular CMs comprised ˜80% of the population (FIG. 6C). CMs were used after day 40 of differentiation, when they assumed an oblong, elongated morphology (FIG. 6C). α-Actinin and MLC2V-eGFP were localised to the Z-disks and A-band respectively, showing that the hiPSC-CMs had a high degree of structural organisation and can recapitulate the human cardiac phenotype.

Human iPSC-CMs were exposed to remdesivir under normal and hypoxic conditions for three days. There is a paucity of data which directly correlates oxygen saturation in vivo with oxygen concentration in vitro. Furthermore, COVID-19 patients exhibit a highly diverse oxygen saturation ranging from normal (>95%), to very low (<70%) (see, Tobin M J, Laghi F, Jubran A. Why COVID-19 Silent Hypoxemia Is Baffling to Physicians. Am J Respir Crit Care Med 2020; 202:356-360), making it difficult to choose a suitable 02 in vitro to represent all patients. Since remdesivir is thought to be most beneficial to patients who receive supplemental oxygen (i.e., mild hypoxia), but not high flow oxygen (severe hypoxia), an oxygen (02) concentration of 2.5% was chosen to simulate mild hypoxia that did not directly cause cell death but may instead potentiate damage induced by external stimuli. Human iPSC-CMs exposed to 2.5% 02 were viable, beat and were morphologically indistinguishable from hiPSC-CMs cultured under normoxic (20% 02) conditions.

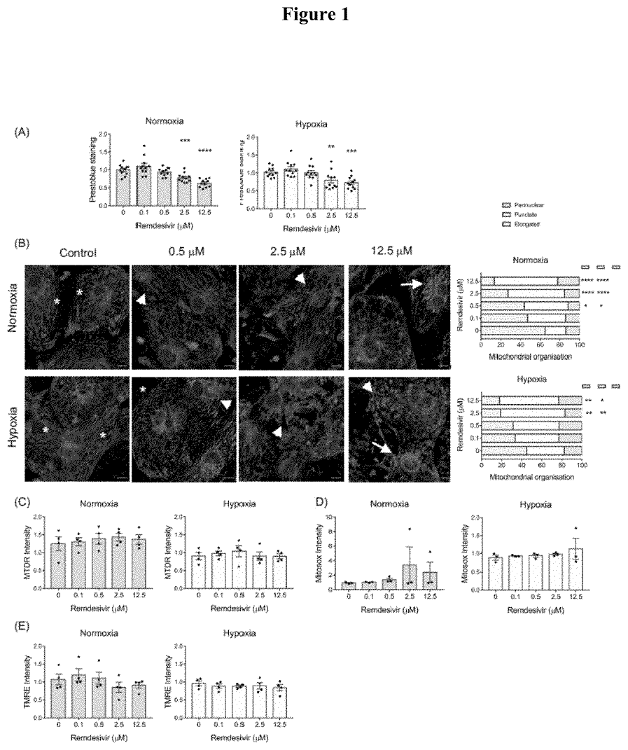

To determine if remdesivir induced mitochondrial dysfunction, the redox potential of hiPSC-CMs was first compared when treated with different doses of this drug under normoxic and hypoxic conditions. The 50% effective concentration (EC50) of remdesivir in vitro is in the (sub)micromolar range while the Cmax of remdesivir in healthy volunteers is 8.9 and 4.3 μM on day 1 and 5 of treatment respectively.³⁸ A dose range of 0.1-12.5 μM was chosen to represent clinically relevant concentrations of remdesivir for cardiotoxicity evaluations. Redox potential is mostly driven by the proton gradient in the mitochondria and is often used as an indicator of metabolic activity in this organelle. Remdesivir dose-dependently reduced redox potential under normoxic and hypoxic conditions (FIG. 1A). While remdesivir had negligible effects at low doses (0.1 μM and 0.5 μM), it significantly decreased redox activity at 2.5 μM and 12.5 μM by 21.0±3.9% and 35.9±4.0% under normoxic, and by 22.5±5.4% and 28.7±6.9% under hypoxic conditions respectively, indicating a repression of cardiac metabolism.

Mitochondrial dynamics constitutes part of the quality control process of this organelle and is maintained by a balance between fusion and fission, which promote the elongation and fragmentation of mitochondria, respectively.^{39, 40} Since mitochondrial dynamics is frequently disturbed by cardiotoxins, it was investigated whether remdesivir altered mitochondrial morphology. Fluorescence imaging revealed three patterns of mitochondrial morphologies among hiPSC-CMs (FIG. 1B). The first consisted of densely organised, elongated mitochondrial networks spread throughout the cytoplasm and this is considered to represent a healthy balance of fission and fusion. The second had punctate, fragmented mitochondria consistent with increased fission. The last had mitochondria located around the nucleus, also suggestive of an imbalance towards increased fission. All hiPSC-CM samples exhibited a mixture of the three phenotypes, but in different proportions. Control, normoxic hiPSC-CMs primarily had an elongated mitochondrial network while remdesivir induced mitochondrial fragmentation in a dose-dependent manner (FIG. 1B). Under normoxic conditions, the proportion of cells with elongated mitochondria significantly decreased from 65.1±6.8% in control hiPSC-CMs to 43.9±7.5%, 27.3±6.0%, and 13.4±5.0% upon treatment with increasing doses of remdesivir at 0.5, 2.5 μM and 12.5 μM respectively, and this was accompanied by a corresponding increase in the proportion of cells with punctate, fragmented mitochondria from 21.0±5.9% to 44.4±7.1, 57.3±7.2 and 64.4±9.5%. Compared to normoxia, hypoxia decreased the prevalence of elongated mitochondria in favour of punctate mitochondria and this worsened with increasing doses of remdesivir. The proportion of elongated mitochondria significantly decreased from 45.1±7.8% in control hPSC-CMs to 18.7±3.0% and 17.8±4.2% upon treatment with 2.5 μM and 12.5 μM of remdesivir, while the proportion of punctate mitochondria rose from 37.3±6.1% in control, to 64.4±4.1% and 58.9±8% at the same doses. The proportion of cells with perinuclear mitochondria was similar in all samples. The mitochondrial mass of hiPSC-CMs, as measured by the intensity of mitochondrial dye staining, was not statistically different among different groups (FIG. 1C), showing that remdesivir promoted mitochondrial fragmentation but did not alter mitochondrial mass in hiPSC-CMs.

To determine if increased oxidative stress contributed to the toxic effects of remdesivir, the level of reactive oxygen species (ROS) in the form of mitochondrial superoxide was measured using the mitosox red dye and was found to be similar in control and treated hiPSC-CMs (FIG. 1D). These results were further confirmed using the CellRox dye, an indicator of ROS in the cytoplasm as well as the mitochondria (data not shown).

It was next tested whether remdesivir induced the depolarisation of mitochondrial membrane potential (Δψm), which is commonly considered to be a prelude to irreversible apoptosis and cell death. No statistically significant decrease in Δψm was detected (FIG. 1E).

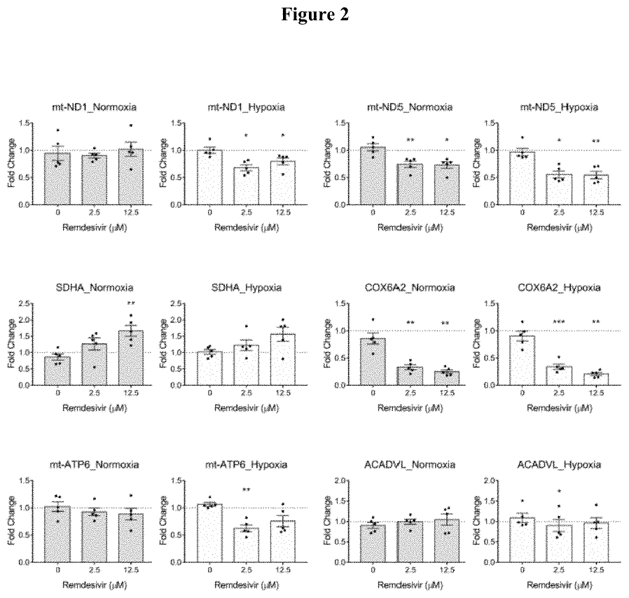

To investigate if remdesivir disturbed the expression of genes important for mitochondrial function, the mRNA levels were measured of genes encoding components of electron transport chain (ETC) encompassing the mitochondrial complex I (ND1 and ND5), II (SDHA), IV (COX6A2) and V (ATP6), as well as a non-ETC gene encoding a mediator of fatty acid β-oxidation in the mitochondria (ACADVL) (FIG. 2). With the exception of SDHA, remdesivir dose-dependently and significantly reduced the expression of ETC genes including ND1, ND5, COX6A2 and ATP6, while ACADVL mRNA levels were similar among all samples. Of the ETC genes, ND1, ND5 and ATP6 are encoded by the mitochondrial genome, while COX6A2 and SDHA are encoded by the nuclear genome. Thus, the effect of remdesivir was not limited to mitochondrial- or nuclear-encoded genes. Overall, the general repression of ETC gene transcripts was consistent with and might contribute to the reduced redox potential observed (FIG. 1A).

### Remdesivir Induced Structural Abnormalities but not Cell Death

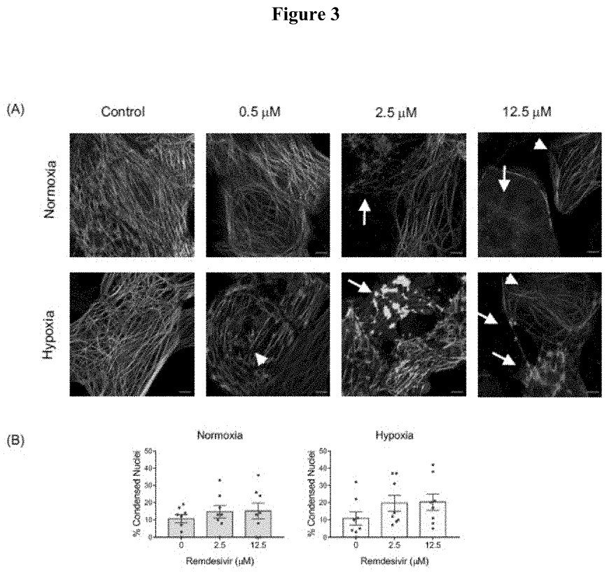

In addition to mitochondrial dysfunction, non-mitochondrial damage was observed in remdesivir-treated cells and manifested as perturbations in sarcomeric arrangement. In control hiPSC-CMs, eGFP-tagged MLC2V assumed a densely-packed striated appearance along myofilaments which spanned the entire cell, reflecting its localisation to the thick filaments of sarcomeres (FIGS. 6 and 3A). Low doses of remdesivir (0.1 and 0.5 μM) did not have any noticeable effect under normoxic conditions, but 0.5 μM of remdesivir under hypoxia induced the thinning and truncation of myofibrils. Human iPSC-CMs treated with 2.5 and 12.5 μM of remdesivir had greatly disorganised myofibrils under both normoxic and hypoxic conditions. While cells with striated structures could still be found, sparse and truncated myofibrils, and patchy MLC2V-eGFP signal with no or poorly discernible organisation were common.

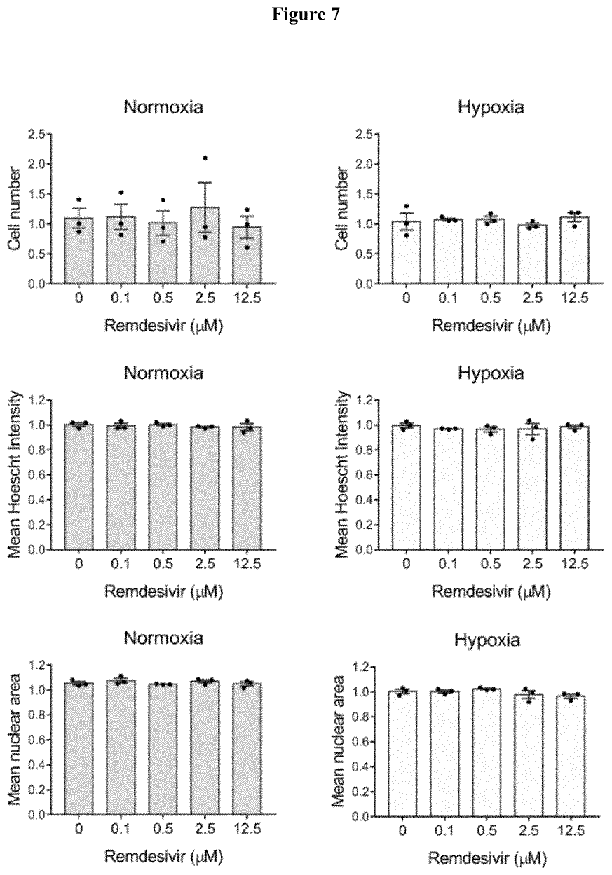

It was next tested if remdesivir induced apoptosis or cell death in hiPSC-CM cultures. Nuclear condensation is a sign of apoptosis and manifest as brightly-stained, small and ‘condensed’ nuclei. Staining with Hoescht nuclear dye did not reveal any significant increase in the number of condensed nuclei following remdesivir treatment (FIG. 3B). Consistently, nuclear area and staining intensity were similar in control and remdesivir treatment groups irrespective of dose and normoxia/hypoxia (FIG. 7). No significant difference in cell number was observed among all samples, demonstrating the absence of significant cell detachment (FIG. 7). It was thus concluded that remdesivir did not significantly induce cell death in hiPSC-CM cultures.

Remdesivir Altered Electrophysiological Properties of hiPSC-CMs

In addition to mitochondrial toxicity, it was also tested if remdesivir disturbed the electrophysiological properties of hiPSC-CMs. Remdesivir (2.5 μM) significantly reduced spontaneous firing frequency (Control 0.89±0.14 vs remdesivir 0.36±0.07 Hz) and the diastolic depolarisation rate (Control 20.32±3.77 vs remdesivir 7.74±1.19 mV/s), and tended to decrease the maximum depolarisation rate (Control 40.94±7.42 vs remdesivir 21.7±6.38 mV/s). The action potential amplitude, and minimal diastolic potential were similar in control and hiPSC-CMs treated with remdesivir.

### Remdesivir Induced Persistent Cardiac Damage

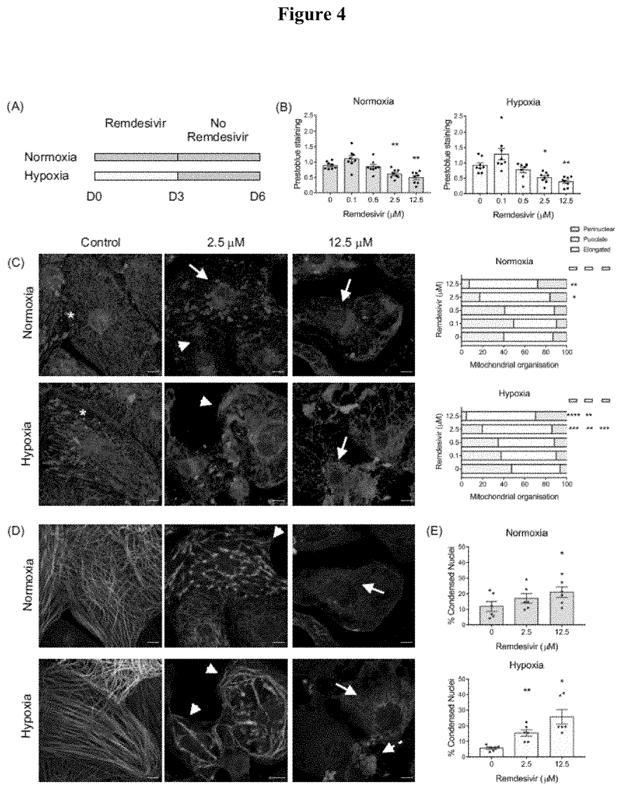

The results so far demonstrated that remdesivir caused mitochondrial, structural, and electrophysiological abnormalities, but significant changes associated with irreversible damage, e.g., A_{W}m depolarisation and nuclear condensation were not observed. The question was therefore if damage induced by remdesivir was transient and reversible. To address this, hiPSC-CMs were exposed to remdesivir as described previously, followed by recovery for 3 days in media devoid of this drug under normoxic conditions to simulate patient recovery after the cessation of remdesivir treatment (FIG. 4A). Remdesivir dose-dependently altered redox potential under normoxic and hypoxic conditions. It significantly decreased redox potential by 31.1±5.3% and 44.1±8.1% at 2.5 μM and 12.5 μM under normoxic condition, and by 41.0±8.7% and 54.6±8.5% under hypoxic conditions (FIG. 4B).

It was also evaluated whether remdesivir induced mitochondrial fragmentation was reversed upon recovery. While low dose of remdesivir (0.5 μM) altered mitochondrial morphology immediately after treatment (FIG. 1B), no significant difference was detected between control and treated cells upon recovery (FIG. 4C). By contrast, the mitochondria of hiPSC-CMs treated with high doses of this drug (2.5 μM and 12.5 μM) remained more fragmented than control. The proportion of cells with elongated mitochondria decreased from 40.1±4.5% in control CMs to 17.2±6.6% and 7.3±3.8% in hiPSC-CMs treated with 2.5 μM and 12.5 μM of remdesivir respectively. Similar trends were observed under hypoxic conditions: showing a decrease in elongated mitochondria from 47.4±4.9% in control to 19.7±2.5% and 4.6±2.1% with 2.5 μM and 12.5 μM remdesivir, and this was accompanied by a corresponding increase in the proportion of cells with punctate mitochondria from 46.1±3.8% to 66.1±1.1% and 65.5±6.6%. The proportion of cells with perinuclear mitochondria increased from 6.5±1.5% in control to 29.9±5.7% with 12.5 μM remdesivir, demonstrating persistent mitochondrial damage (FIGS. 1B and 4C). Apart from mitochondrial changes, structural alterations also persisted beyond the three-day treatment period. Cells treated with 2.5 μM and 12.5 μM of remdesivir showed disorganised sarcomeric arrangement analogous to the patterns seen immediately after treatment (FIGS. 3A and 4D).

Lastly, the prevalence of nuclear condensation was evaluated as a surrogate for apoptosis and cell death. Although no increase in nuclear condensation was seen immediately after three days of treatment (FIG. 2B), a dose dependent increase in the proportion of condensed nuclei was observed in hiPSC-CMs three days after they were treated with 2.5 μM and 12.5 μM of remdesivir (FIG. 4E).

In summary, remdesivir induced mitochondrial and structural damage, which persisted beyond the treatment period, culminating in increased cell death.

### Remdesivir-Induced Cardiotoxicity is Ameliorated by an Inhibitor of Mitochondrial Fission

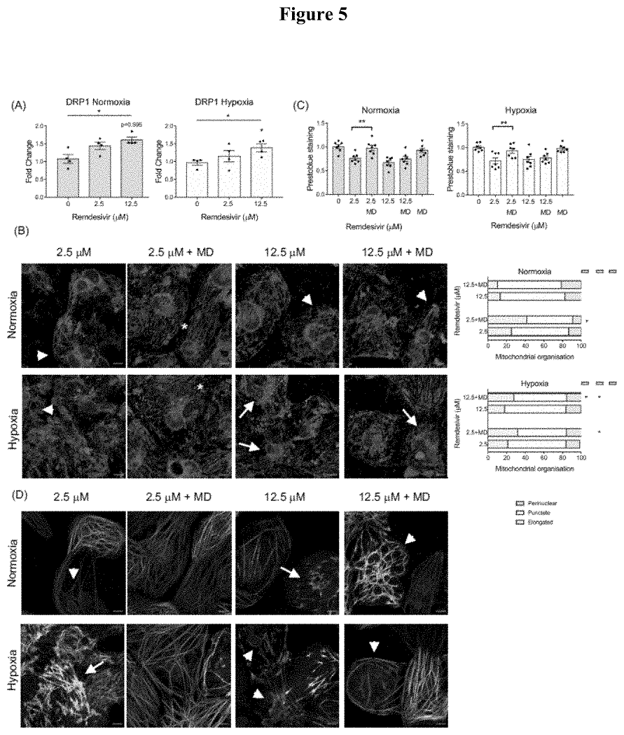

Given the extensive mitochondrial fragmentation observed in remdesivir treated hiPSC-CMs, it was proposed that increased mitochondrial fission might underlie the cardiotoxic effects induced by this drug. To test this, the level of DRP1, a master regulator of mitochondrial fission,^{40, 41} was measured, and a dose-dependent and significant increase was identified in cells treated with remdesivir (FIG. 5A). These results were consistent with an imbalance towards mitochondrial fission, possibly driven by increased DRP1 expression. Next, it was posited that the inhibition of DRP1 could protect against remdesivir and applied mdivi-1, which is a well-established inhibitor of DRP1⁴², to hiPSC-CMs. Co-treatment of mdivi-1 with 2.5 μM remdesivir significantly promoted the formation of elongated mitochondria under normoxic conditions compared to cells treated with remdesivir alone (FIG. 5B). Mdivi-1 treatment also produced a corresponding decrease in the proportion of cells with punctate mitochondrial under hypoxia. However, at 12.5 μM remdesivir, mdivi-1 could only significantly protect hiPSC-CMs under hypoxic but not normoxic conditions.

It was next tested if preventing mitochondrial fragmentation with mdivi-1 could normalise redox potential in hPSC-CMs. Under both normoxic and hypoxic conditions, mdivi-1 restored redox potential in hiPSC-CMs treated with 2.5 μM of remdesivir to control levels (FIG. 5C). However, the same trend was not observed at 12.5 μM of remdesivir, at which mdivi-1 did not have noticeable effect.

Lastly, it was shown that the structural abnormalities induced by remdesivir could be ameliorated by mdivi-1. Mdivi-1 improved the sarcomere arrangement of CMs treated with 2.5 μM, and 12.5 μM of remdesivir (FIG. 5D). Myofibrils were more densely packed, and MLC2V-eGFP signal was more striated with mdivi-1 co-treatment than remdesivir alone.

These results showed that remdesivir induced cardiotoxicity could be ameliorated by cardioprotective strategies in vitro and identified excessive mitochondrial fission to be a mechanistic contributor to this phenotype.

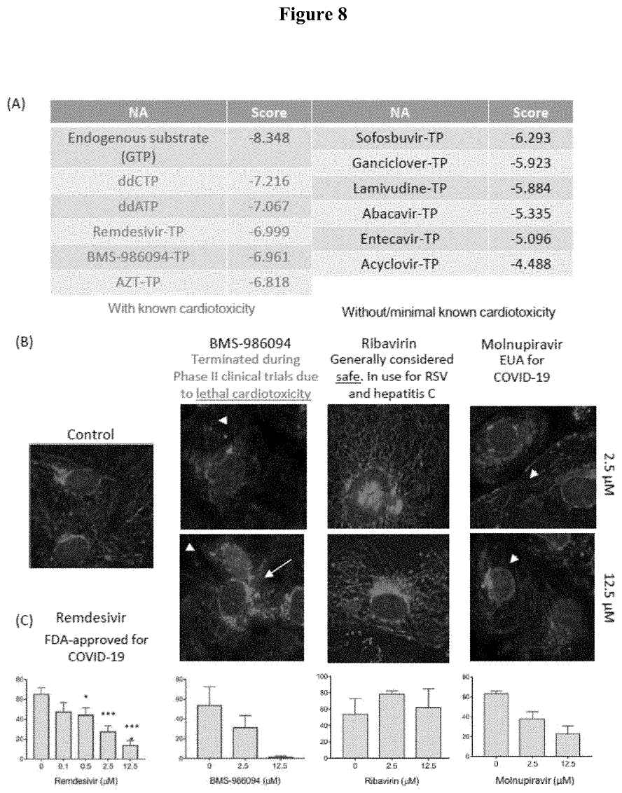

Molecular docking revealed higher affinity interactions between DRP1 and nucleotide analogues known to cause toxicity (FIG. 8A). The strong correlation between binding affinity with known toxicity demonstrated the predictive power of this method. Experimental validation with other NAs also demonstrated increased mitochondrial fragmentation, indicating the latter as a general feature of these compounds (FIG. 8B-C).

### DISCUSSION

Remdesivir is a nucleotide analogue effective against COVID-19, but its potential to cause cardiotoxicity is unclear. Using an in vitro hiPSC-CM model, the present inventors showed that remdesivir induced cardiotoxicity at clinically relevant concentrations under normoxic and hypoxic conditions. Mitochondrial dysfunctions, in the form of mitochondrial fragmentation and reduced redox potential, and sarcomere disarray were detected, and they persisted after cessation of treatment. This is the first report of adverse and persistent cardiac effects associated with remdesivir at clinically relevant concentrations and highlights the importance of cardiac monitoring in patients treated with this drug. Importantly, inhibition of mitochondrial fission protected against mitochondrial fragmentation, normalised redox potential and prevented sarcomere disarray, indicating that remdesivir induced cardiotoxicity can be ameliorated.

The efficacy of remdesivir against COVID-19 has been demonstrated in many in vitro and in vivo studies.^{2, 3} Remdesivir is effective in vitro in the sub-micromolar range: it blocked SARS-CoV-2 infection in African green monkey kidney Vero E6 cells with an EC50 and EC90 of 0.77 μM and 1.76 μM respectively,³ and in human airway epithelial cells, at an IC50 of 0.069 μM.⁴³ Remdesivir is also efficacious in randomised clinical trials and shortened time to recovery.⁵ In light of these findings, remdesivir received authorisation from the US FDA for use in hospitalised patients with COVID-19 with a standard dose of 200 mg, followed by daily doses of 100 mg for 4 or 9 days. In healthy adult volunteers receiving a similar dose regimen (200 mg on day 1, 100 mg on days 2-4), and the peak plasma concentrations were 5.4 μg/mL (8.9 μM) on day 1 and 2.6 μg/mL (4.3 μM) on day 5.^{38, 44, 45} Thus both the EC50 and the Cmax of remdesivir are within the dosage range tested in this study (0.1-12.5 μM). Increased mitochondrial fragmentation at 0.5 μM was first detected, but these perturbations did not persist. On the other hand, multiple features of mitochondrial and sarcomeric abnormalities were observed at ≥2.5 μM under normoxic and hypoxic conditions, which is 3.6 and 1.7 fold below Cmax in patients on day 1 and 5 of treatment. Importantly, these abnormalities persisted after cessation of treatment, culminating in increased cell death.

Mitochondrial toxicity of remdesivir has recently been demonstrated in two human intestinal (HT29 and HCT116) and two human liver cell lines (HepG2 and PLC/PRF/5) at high doses of 10 or 20 μM⁴⁶ Remdesivir was also shown to prolong QT intervals in hPSC-CMs.³⁵ This disclosure is the first report of persistent toxicity in CMs at clinically relevant concentrations several folds below Cmax. By systematically evaluating the mitochondrial phenotype of remdesivir treated cells, the present inventors showed that remdesivir primarily induced mitochondrial fragmentation and reduced redox potential. This phenotype was found to be similar under normoxic and hypoxic conditions. Mitochondrial damage may occur via various mechanisms. The toxicity of ribonucleoside/nucleotide analogues has largely been attributed to inhibition of POLRMT¹², the RNA polymerase responsible for the transcription of mitochondria DNA. However, purified human mitochondrial RNA polymerase has been shown to effectively discriminate against remdesivir-triphosphate with a selectivity value of ˜500-fold,⁴⁷ suggesting that inhibition of POLRMT is unlikely to be a major contributor of the cardiotoxic effects of remdesivir. Instead, excessive mitochondrial fission was identified to be a critical driver of the remdesivir induced abnormalities, since the inhibition of the former with a small molecule inhibitor, mdivi-1, reduced mitochondrial fragmentation, normalised redox potential and improved sarcomeric arrangement in cells treated with 2.5 μM of remdesivir. Excessive mitochondrial has previously been shown to contribute to a range of cardiac disorders and mdivi-1 is cardioprotective in the settings of doxorubicin induced cardiotoxicity,⁴⁸ ischemia-reperfusion injury,^{49, 50} and pressure overload induced heart failure.⁵¹ This disclosure provides important proof-of-principle that cardioprotective strategies can be employed to at least partially protect CMs against the cardiotoxic effects of remdesivir. It is possible that additional factors contribute to the cardiotoxicity of remdesivir at extremely high doses and this may explain the lesser ability of mdivi-1 to protect against remdesivir at 12.5 μM.

In addition to mitochondrial damage, single cell patch clamp analysis revealed electrophysiological alterations in hiPSC-CMs treated with 2.5 μM of remdesivir. Specifically, remdesivir decreased firing frequency, consistent with a previous examination of remdesivir using MEA. Both these observations are in line with recent reports of bradycardia observed in patients (see, Touafchia A, Bagheri H, Carrie D, Durrieu G, Sommet A, Chouchana L, Montastruc F. Serious bradycardia and remdesivir for coronavirus 2019 (COVID-19): a new safety concerns. Clinical microbiology and infection: the official publication of the European Society of Clinical Microbiology and Infectious Diseases 2021).

These demonstrations of cardiotoxicity were unexpected, given that the use of remdesivir in animal models and in patients is generally considered to be safe. Remdesivir has been associated with a slight increase in the risk of cardiac arrest (1.9% vs 1.4%) and serious atrial fibrillation (0.9% vs 0.2%) compared to placebo in one clinical study⁵, but the Solidarity trial by the WHO showed similar rates of cardiac death (0.3% vs 0.4%).⁷ Adverse cardiovascular events and evaluations of cardiac stress markers were not reported in other clinical studies.^{52, 53} Due to the low numbers of cardiovascular incidences reported, it is unclear whether the difference in cardiac outcome observed between remdesivir and placebo groups are significant, but immediate, overt cardiotoxicity was not apparent. There are potentially multiple reasons for the dichotomy between this study and currently available clinical data. Firstly, in vitro derived hPSC-CMs were utilised, such cells may be immature and may not respond in the same manner as human adult CMs.^{32, 33, 54} Therefore, further investigations in animal models or in patients would be necessary to confirm cardiotoxicity in vivo. Secondly, the cardiotoxicity of remdesivir initially manifests as mitochondrial and structural abnormalities without overt cell death during the treatment period, and may not be immediately apparent in clinical trials, which primarily report acute clinical adverse events. Thirdly, pre-existing cardiac co-morbidities, and direct cardiac damage caused by COVID-19 may mask any adverse effects of remdesivir.

Remdesivir is currently used clinically at a concentration that far exceeds its effective concentration in vitro. Remdesivir can inhibit SARS-CoV-2 in African green monkey kidney Vero E6 and human airway epithelial cells with an EC/IC50 of 0.77 μM and 0.069 μM. respectively^{3, 43}. Similarly, recent studies in hPSC-CMs also demonstrated efficacy of remdesivir at 0.2-0.6 μM.^{23, 35} While remdesivir induced cardiotoxicity ≤2.5 μM, low concentration of this drug closer to its in vitro effective dose (0.1 μM and 0.5 μM) did not induce persistent functional alterations. It may be beneficial to assess the in vivo efficacy of this drug at lower doses to minimise adverse effects. Consistent with this, clinical trials comparing 5 or 10 days of remdesivir treatment either showed that there was no statistically significant difference in clinical outcome, or that the 5-day regimen was slightly superior.^{52, 53} A careful titration of dose and duration may help to balance antiviral efficacy with cardiotoxic risks.

Despite the limitations of The animal model used in this study, the demonstration of persistent cardiotoxicity at clinically relevant concentrations highlight the need for further investigations of cardiotoxicity in in vivo animal models, and clinical studies. Adult CMs have limited ability to regenerate, thus treatment-induced cardiotoxicity can potentially cause irreparable harm to patients already made vulnerable by cardiovascular co-morbidities and cardiac damage caused by viral infection. Functional cardiac monitoring in the clinic such as troponin release and MRI measurements are warranted such that cardiotoxicity does not contribute to the long term sequelae of COVID-19 patients.

All patents, patent applications, and other publications, including GenBank Accession Numbers and equivalents, cited in this application are incorporated by reference in the entirety for all purposes.

TABLE 1
Primer Sequences for qRT-PCR

| Target | | |
|---|---|---|
| Gene | Forward (5′ to 3′) | Reverse (5′ to 3′) |
| B2M | CCACTGAAAAAGATGAGTATGCCT | CCAATCCAAATGCGGCATCTTCA |
| MT-ND1 | CCACCTCTAGCCTAGCCGTTTA | GGGTCATGATGGCAGGAGTAAT |
| MT-ND5 | ACTAAACCCCATTAAACGCC | TTGAGGTCTAGGGCTGTTAG |
| SDHA | GTTCGACCGGGGAATGGTC | CCGCACCTTGTAGTCTTCCC |
| COX6A2 | GCATCCGCACCAAGCCCTA | GAGGGTTCACGTGGCTATTG |
| MT-ATP6 | CAACAACCGACTAATCACCA | GGGTGGTTGGTGTAAATGAG |
| ACADVL | CTTTGCAACACCCAGTACGC | GCAGGATGCCTTTGAAACCG |
| DRP1 | GATGCCATAGTTGAAGTGGTGAC | CCACAAGCATCAOCAAAGTCTGG |

UniProtKB-O00429
SEQ ID NO: 1
MEALIPVINK LQDVFNTVGA DIIQLPQIVV VGTQSSGKSS VLESLVGRDL LPRGTGIVTR
RPLILQLVHV SQEDKRKTTG EENGVEAEEW GKFLHTKNKL YTDFDEIRQE IENETERISG
NNKGVSPEPI HLKIFSPNVV NLTLVDLPGM TKVPVGDQPK DIELQIRELI LRFISNPNSI
ILAVTAANTD MATSEALKIS REVDPDGRRT LAVITKLDLM DAGTDAMDVL MGRVIPVKLG
IIGVVNRSQL DINNKKSVTD SIRDEYAFLQ KKYPSLANRN GTKYLARTLN RLLMHHIRDC
LPELKTRINV LAAQYQSLLN SYGEPVDDKS ATLLQLITKF ATEYCNTIEG TAKYIETSEL
CGGARICYIF HETFGRTLES VDPLGGLNTI DILTAIRNAT GPRPALFVPE VSFELLVKRQ
IKRLEEPSLR CVELVHEEMQ RIIQHCSNYS TQELLRFPKL HDAIVEVVTC LLRKRLPVTN
EMVHNLVAIE LAYINTKHPD FADACGLMNN NIEEQRRNRL ARELPSAVSR DKSSKVPSAL
APASQEPSPA ASAEADGKLI QDSRRETKNV ASGGGGVGDG VQEPTTGNWR GMLKTSKAEE
LLAEEKSKPI PIMPASPQKG HAVNLLDVPV PVARKLSARE QRDCEVIERL IKSYFLIVRK
NIQDSVPKAV MHFLVNHVKD TLQSELVGQL YKSSLLDDLL TESEDMAQRR KEAADMLKAL
QGASQIIAEI RETHLW

### REFERENCES

1. Madjid M, Safavi-Naeini P, Solomon S D, Vardeny O. Potential Effects of Coronaviruses on the Cardiovascular System: A Review. JAMA Cardiol 2020.
2. Williamson B N, Feldmann F, Schwarz B, Meade-White K, Porter D P, Schulz J, van Doremalen N, Leighton I, Yinda C K, Perez-Perez L, Okumura A, Lovaglio J, Hanley P W, Saturday G, Bosio C M, Anzick S, Barbian K, Cihlar T, Martens C, Scott D P, Munster V J, de Wit E. Clinical benefit of remdesivir in rhesus macaques infected with SARS-CoV-2. Nature 2020.
3. Wang M, Cao R, Zhang L, Yang X, Liu J, Xu M, Shi Z, Hu Z, Zhong W, Xiao G. Remdesivir and chloroquine effectively inhibit the recently emerged novel coronavirus (2019-nCoV) in vitro. Cell Res 2020; 30:269-271.
4. Pruijssers A J, George A S, Schafer A, Leist S R, Gralinksi L E, Dinnon K H, 3rd, Yount B L, Agostini M L, Stevens L J, Chappell J D, Lu X, Hughes T M, Gully K, Martinez D R, Brown A J, Graham R L, Perry J K, Du Pont V, Pitts J, Ma B, Babusis D, Murakami E, Feng J Y, Bilello J P, Porter D P, Cihlar T, Baric R S, Denison M R, Sheahan T P. Remdesivir Inhibits SARS-CoV-2 in Human Lung Cells and Chimeric SARS-CoV Expressing the SARS-CoV-2 RNA Polymerase in Mice. Cell reports 2020; 32:107940.
5. Beigel J H, Tomashek K M, Dodd L E, Mehta A K, Zingman B S, Kalil A C, Hohmann E, Chu H Y, Luetkemeyer A, Kline S, Lopez de Castilla D, Finberg R W, Dierberg K, Tapson V, Hsieh L, Patterson T F, Paredes R, Sweeney D A, Short W R, Touloumi G, Lye D C, Ohmagari N, Oh M D, Ruiz-Palacios G M, Benfield T, Fatkenheuer G, Kortepeter M G, Atmar R L, Creech C B, Lundgren J, Babiker A G, Pett S, Neaton J D, Burgess T H, Bonnett T, Green M, Makowski M, Osinusi A, Nayak S, Lane H C, Members A-SG. Remdesivir for the Treatment of Covid-19—Final Report. N Engl J Med 2020.
6. Wang Y, Zhang D, Du G, Du R, Zhao J, Jin Y, Fu S, Gao L, Cheng Z, Lu Q, Hu Y, Luo G, Wang K, Lu Y, Li H, Wang S, Ruan S, Yang C, Mei C, Wang Y, Ding D, Wu F, Tang X, Ye X, Ye Y, Liu B, Yang J, Yin W, Wang A, Fan G, Zhou F, Liu Z, Gu X, Xu J, Shang L, Zhang Y, Cao L, Guo T, Wan Y, Qin H, Jiang Y, Jaki T, Hayden F G, Horby P W, Cao B, Wang C. Remdesivir in adults with severe COVID-19: a randomised, double-blind, placebo-controlled, multicentre trial. Lancet 2020; 395:1569-1578.
7. Consortium WHOST, Pan H, Peto R, Henao-Restrepo A M, Preziosi M P, Sathiyamoorthy V, Abdool Karim Q, Alejandria M M, Hernandez Garcia C, Kieny M P, Malekzadeh R, Murthy S, Reddy K S, Roses Periago M, Abi Hanna P, Ader F, Al-Bader A M, Alhasawi A, Allum E, Alotaibi A, Alvarez-Moreno C A, Appadoo S, Asiri A, Aukrust P, Barratt-Due A, Bellani S, Branca M, Cappel-Porter H B C, Cerrato N, Chow T S, Como N, Eustace J, Garcia P J, Godbole S, Gotuzzo E, Griskevicius L, Hamra R, Hassan M, Hassany M, Hutton D, Irmansyah I, Jancoriene L, Kirwan J, Kumar S, Lennon P, Lopardo G, Lydon P, Magrini N, Maguire T, Manevska S, Manuel O, McGinty S, Medina M T, Mesa Rubio M L, Miranda-Montoya M C, Nel J, Nunes E P, Perola M, Portoles A, Rasmin M R, Raza A, Rees H, Reges P P S, Rogers C A, Salami K, Salvadori M I, Sinani N, Sterne J A C, Stevanovikj M, Tacconelli E, Tikkinen K A O, Trelle S, Zaid H, Rottingen J A, Swaminathan S. Repurposed Antiviral Drugs for Covid-19—Interim WHO Solidarity Trial Results. N Engl J Med 2020.
8. FDA Approves First Treatment for COVID-19. 2020.
9. Yin W, Mao C, Luan X, Shen D D, Shen Q, Su H, Wang X, Zhou F, Zhao W, Gao M, Chang S, Xie Y C, Tian G, Jiang H W, Tao S C, Shen J, Jiang Y, Jiang H, Xu Y, Zhang S, Zhang Y, Xu H E. Structural basis for inhibition of the RNA-dependent RNA polymerase from SARS-CoV-2 by remdesivir. Science 2020; 368:1499-1504.
10. Apostolova N, Blas-Garcia A, Esplugues J V. Mitochondrial interference by anti-HIV drugs: mechanisms beyond Pol-gamma inhibition. Trends in pharmacological sciences 2011; 32:715-725.
11. Group DADS, Sabin C A, Worm S W, Weber R, Reiss P, El-Sadr W, Dabis F, De Wit S, Law M, D′Arminio Monforte A, Friis-Moller N, Kirk O, Pradier C, Weller I, Phillips A N, Lundgren J D. Use of nucleoside reverse transcriptase inhibitors and risk of myocardial infarction in HIV-infected patients enrolled in the D:A:D study: a multi-cohort collaboration. Lancet 2008; 371:1417-1426.
12. Lewis W, Simpson J F, Meyer R R. Cardiac mitochondrial DNA polymerase-gamma is inhibited competitively and noncompetitively by phosphorylated zidovudine. Circ Res 1994; 74:344-348.
13. Nomura R, Sato T, Sato Y, Medin J A, Kushimoto S, Yanagisawa T. Azidothymidine-triphosphate impairs mitochondrial dynamics by disrupting the quality control system. Redox Biol 2017; 13:407-417.
14. Lund K C, Wallace K B. Adenosine 3′,5′-cyclic monophosphate (cAMP)-dependent phosphoregulation of mitochondrial complex I is inhibited by nucleoside reverse transcriptase inhibitors. Toxicol Appl Pharmacol 2008; 226:94-106.
15. Gao R Y, Mukhopadhyay P, Mohanraj R, Wang H, Horvath B, Yin S, Pacher P. Resveratrol attenuates azidothymidine-induced cardiotoxicity by decreasing mitochondrial reactive oxygen species generation in human cardiomyocytes. Molecular medicine reports 2011; 4:151-155.
16. Bansal M. Cardiovascular disease and COVID-19. Diabetes Metab Syndr 2020; 14:247-250.
17. Zhou F, Yu T, Du R, Fan G, Liu Y, Liu Z, Xiang J, Wang Y, Song B, Gu X, Guan L, Wei Y, Li H, Wu X, Xu J, Tu S, Zhang Y, Chen H, Cao B. Clinical course and risk factors for mortality of adult inpatients with COVID-19 in Wuhan, China: a retrospective cohort study. Lancet 2020; 395:1054-1062.
18. Shi S, Qin M, Shen B, Cai Y, Liu T, Yang F, Gong W, Liu X, Liang J, Zhao Q, Huang H, Yang B, Huang C. Association of Cardiac Injury With Mortality in Hospitalized Patients With COVID-19 in Wuhan, China. JAMA Cardiol 2020.
19. Sharma A, Garcia G, Jr., Wang Y, Plummer J T, Morizono K, Arumugaswami V, Svendsen C N. Human iPSC-Derived Cardiomyocytes Are Susceptible to SARS-CoV-2 Infection. Cell Rep Med 2020; 1:100052.
20. Yang L, Han Y, Nilsson-Payant B E, Gupta V, Wang P, Duan X, Tang X, Zhu J, Zhao Z, Jaffre F, Zhang T, Kim T W, Harschnitz O, Redmond D, Houghton S, Liu C, Naji A, Ciceri G, Guttikonda S, Bram Y, Nguyen D T, Cioffi M, Chandar V, Hoagland D A, Huang Y, Xiang J, Wang H, Lyden D, Borczuk A, Chen H J, Studer L, Pan F C, Ho D D, tenOever B R, Evans T, Schwartz R E, Chen S. A Human Pluripotent Stem Cell-based Platform to Study SARS-CoV-2 Tropism and Model Virus Infection in Human Cells and Organoids. Cell Stem Cell 2020; 27:125-136 e127.
21. Wichmann D, Sperhake J P, Lutgehetmann M, Steurer S, Edler C, Heinemann A, Heinrich F, Mushumba H, Kniep I, Schroder A S, Burdelski C, de Heer G, Nierhaus A, Frings D, Pfefferle S, Becker H, Bredereke-Wiedling H, de Weerth A, Paschen H R, Sheikhzadeh-Eggers S, Stang A, Schmiedel S, Bokemeyer C, Addo M M, Aepfelbacher M, Puschel K, Kluge S. Autopsy Findings and Venous Thromboembolism in Patients With COVID-19: A Prospective Cohort Study. Annals of internal medicine 2020; 173:268-277.
22. Bose R J C, McCarthy J R. Direct SARS-CoV-2 infection of the heart potentiates the cardiovascular sequelae of COVID-19. Drug Discov Today 2020.
23. Bojkova D, Wagner J U G, Shumliakivska M, Aslan G S, Saleem U, Hansen A, Luxan G, Gunther S, Pham M D, Krishnan J, Harter P N, Ermel U H, Frangakis A S, Milting H, Zeiher A M, Klingel K, Cinatl J, Dendorfer A, Eschenhagen T, Tschope C, Ciesek S, Dimmeler S. SARS-CoV-2 infects and induces cytotoxic effects in human cardiomyocytes. Cardiovasc Res 2020.
24. Gopal R, Marinelli M A, Alcorn J F. Immune Mechanisms in Cardiovascular Diseases Associated With Viral Infection. Frontiers in immunology 2020; 11:570681.
25. Magadum A, Kishore R. Cardiovascular Manifestations of COVID-19 Infection. Cells 2020; 9.
26. Xie J, Covassin N, Fan Z, Singh P, Gao W, Li G, Kara T, Somers V K. Association Between Hypoxemia and Mortality in Patients With COVID-19. Mayo Clinic proceedings Mayo Clinic 2020; 95:1138-1147.
27. Karbalai Saleh S, Oraii A, Soleimani A, Hadadi A, Shajari Z, Montazeri M, Moradi H, Talebpour M, Sadat Naseri A, Balali P, Akhbari M, Ashraf H. The association between cardiac injury and outcomes in hospitalized patients with COVID-19. Internal and emergency medicine 2020; 15:1415-1424.
28. Musunuru K, Sheikh F, Gupta R M, Houser S R, Maher K O, Milan D J, Terzic A, Wu J C, American Heart Association Council on Functional G, Translational B, Council on Cardiovascular Disease in the Y, Council on C, Stroke N. Induced Pluripotent Stem Cells for Cardiovascular Disease Modeling and Precision Medicine: A Scientific Statement From the American Heart Association. Circ Genom Precis Med 2018; 11:e000043.
29. Gintant G, Burridge P, Gepstein L, Harding S, Herron T, Hong C, Jalife J, Wu J C. Use of Human Induced Pluripotent Stem Cell-Derived Cardiomyocytes in Preclinical Cancer Drug Cardiotoxicity Testing: A Scientific Statement From the American Heart Association. Circ Res 2019; 125:e75-e92.
30. Magdy T, Schuldt A J T, Wu J C, Bernstein D, Burridge P W. Human Induced Pluripotent Stem Cell (hiPSC)-Derived Cells to Assess Drug Cardiotoxicity: Opportunities and Problems. Annu Rev Pharmacol Toxicol 2018; 58:83-103.
31. Paik D T, Chandy M, Wu J C. Patient and Disease-Specific Induced Pluripotent Stem Cells for Discovery of Personalized Cardiovascular Drugs and Therapeutics. Pharmacological reviews 2020; 72:320-342.
32. Poon E, Yan B, Zhang S, Rushing S, Keung W, Ren L, Lieu D K, Geng L, Kong C W, Wang J, Wong H S, Boheler K R, Li R A. Transcriptome-guided functional analyses reveal novel biological properties and regulatory hierarchy of human embryonic stem cell-derived ventricular cardiomyocytes crucial for maturation. PLoS One 2013; 8:e77784.
33. Poon E N, Hao B, Guan D, Jun Li M, Lu J, Yang Y, Wu B, Wu S C, Webb S E, Liang Y, Miller A L, Yao X, Wang J, Yan B, Boheler K R. Integrated transcriptomic and regulatory network analyses identify microRNA-200c as a novel repressor of human pluripotent stem cell-derived cardiomyocyte differentiation and maturation. Cardiovasc Res 2018; 114:894-906.
34. Poon E N, Luo X L, Webb S E, Yan B, Zhao R, Wu S C M, Yang Y, Zhang P, Bai H, Shao J, Chan C M, Chan G C, Tsang S Y, Gundry R L, Yang H T, Boheler K R. The cell surface marker CD36 selectively identifies matured, mitochondria-rich hPSC-cardiomyocytes. Cell Res 2020; 30:626-629.
35. Choi S W, Shin J S, Park S J, Jung E, Park Y G, Lee J, Kim S J, Park H J, Lee J H, Park S M, Moon S H, Ban K, Go Y Y. Antiviral activity and safety of remdesivir against SARS-CoV-2 infection in human pluripotent stem cell-derived cardiomyocytes. Antiviral Res 2020:104955.
36. Wong C K, Luk H K, Lai W H, Lau Y M, Zhang R R, Wong A C, Lo G C, Chan K H, Hung I F, Tse H F, Woo P C, Lau S K, Siu C W. Human-Induced Pluripotent Stem Cell-Derived Cardiomyocytes Platform to Study SARS-CoV-2 Related Myocardial Injury. Circulation journal: official journal of the Japanese Circulation Society 2020.
37. Yang X, Rodriguez M L, Leonard A, Sun L, Fischer K A, Wang Y, Ritterhoff J, Zhao L, Kolwicz S C, Jr., Pabon L, Reinecke H, Sniadecki N J, Tian R, Ruohola-Baker H, Xu H, Murry C E. Fatty Acids Enhance the Maturation of Cardiomyocytes Derived from Human Pluripotent Stem Cells. Stem cell reports 2019; 13:657-668.
38. Investigator's brochure of remdesivir. In: Sciences G, ed., 5th edition ed.
39. Ni H M, Williams J A, Ding W X. Mitochondrial dynamics and mitochondrial quality control. Redox Biol 2015; 4:6-13.
40. Archer S L. Mitochondrial dynamics—mitochondrial fission and fusion in human diseases. N Engl J Med 2013; 369:2236-2251.
41. Smirnova E, Griparic L, Shurland D L, van der Bliek A M. Dynamin-related protein Drpl is required for mitochondrial division in mammalian cells. Mol Biol Cell 2001; 12:2245-2256.
42. Cassidy-Stone A, Chipuk J E, Ingerman E, Song C, Yoo C, Kuwana T, Kurth M J, Shaw J T, Hinshaw J E, Green D R, Nunnari J. Chemical inhibition of the mitochondrial division dynamin reveals its role in Bax/Bak-dependent mitochondrial outer membrane permeabilization. Developmental cell 2008; 14:193-204.
43. Sheahan T P, Sims A C, Graham R L, Menachery V D, Gralinski L E, Case J B, Leist S R, Pyrc K, Feng J Y, Trantcheva I, Bannister R, Park Y, Babusis D, Clarke M O, Mackman R L, Spahn J E, Palmiotti C A, Siegel D, Ray A S, Cihlar T, Jordan R, Denison M R, Baric R S. Broad-spectrum antiviral GS-5734 inhibits both epidemic and zoonotic coronaviruses. Sci Transl Med 2017; 9.
44. Agency E M. Summary on compassionate use. Remdesivir Gilead. Product No. EMEA/H/K/005622/CU., 2020.
45. Jorgensen S C J, Kebriaei R, Dresser L D. Remdesivir: Review of Pharmacology, Pre-clinical Data, and Emerging Clinical Experience for COVID-19. Pharmacotherapy 2020; 40:659-671.
46. Akinci E, Cha M, Lin L, Yeo G, Hamilton M C, Donahue C J, Bermudez-Cabrera H C, Zanetti L C, Chen M, Barkal S A, Khowpinitchai B, Chu N, Velimirovic M, Jodhani R, Fife J D, Sovrovic M, Cole P A, Davey R A, Cassa C A, Sherwood R I. Elucidation of remdesivir cytotoxicity pathways through genome-wide CRISPR-Cas9 screening and transcriptomics. bioRxiv 2020.
47. Tchesnokov E P, Feng J Y, Porter D P, Gotte M. Mechanism of Inhibition of Ebola Virus RNA-Dependent RNA Polymerase by Remdesivir. Viruses 2019; 11.
48. Gharanei M, Hussain A, Janneh O, Maddock H. Attenuation of doxorubicin-induced cardiotoxicity by mdivi-1: a mitochondrial division/mitophagy inhibitor. PLoS One 2013; 8:e77713.
49. Ishikita A, Matoba T, Ikeda G, Koga J, Mao Y, Nakano K, Takeuchi O, Sadoshima J, Egashira K. Nanoparticle-Mediated Delivery of Mitochondrial Division Inhibitor 1 to the Myocardium Protects the Heart From Ischemia-Reperfusion Injury Through Inhibition of Mitochondria Outer Membrane Permeabilization: A New Therapeutic Modality for Acute Myocardial Infarction. Journal of the American Heart Association 2016; 5.
50. Maneechote C, Palee S, Kerdphoo S, Jaiwongkam T, Chattipakorn S C, Chattipakorn N. Differential temporal inhibition of mitochondrial fission by Mdivi-1 exerts effective cardioprotection in cardiac ischemia/reperfusion injury. Clinical science 2018; 132:1669-1683.
51. Givvimani S, Munjal C, Tyagi N, Sen U, Metreveli N, Tyagi S C. Mitochondrial division/mitophagy inhibitor (Mdivi) ameliorates pressure overload induced heart failure. PLoS One 2012; 7:e32388.
52. Spinner C D, Gottlieb R L, Criner G J, Arribas Lopez J R, Cattelan A M, Soriano Viladomiu A, Ogbuagu O, Malhotra P, Mullane K M, Castagna A, Chai L Y A, Roestenberg M, Tsang O T Y, Bemasconi E, Le Tumier P, Chang S C, SenGupta D, Hyland R H, Osinusi A O, Cao H, Blair C, Wang H, Gaggar A, Brainard D M, McPhail M J, Bhagani S, Ahn M Y, Sanyal A J, Huhn G, Marty F M, Investigators G-U-. Effect of Remdesivir vs Standard Care on Clinical Status at 11 Days in Patients With Moderate COVID-19: A Randomized Clinical Trial. Jama 2020; 324:1048-1057.
53. Goldman J D, Lye D C B, Hui D S, Marks K M, Bruno R, Montejano R, Spinner C D, Galli M, Ahn M Y, Nahass R G, Chen Y S, SenGupta D, Hyland R H, Osinusi A O, Cao H, Blair C, Wei X, Gaggar A, Brainard D M, Towner W J, Munoz J, Mullane K M, Marty F M, Tashima K T, Diaz G, Subramanian A, Investigators G-U-. Remdesivir for 5 or 10 Days in Patients with Severe Covid-19. N Engl Med 2020.
54. Poon E, Keung W, Liang Y, Ramalingam R, Yan B, Zhang S, Chopra A, Moore J, Herren A, Lieu D K, Wong H S, Weng Z, Wong O T, Lam Y W, Tomaselli G F, Chen C, Boheler K R, Li R A. Proteomic Analysis of Human Pluripotent Stem Cell-Derived, Fetal, and Adult Ventricular Cardiomyocytes Reveals Pathways Crucial for Cardiac Metabolism and Maturation. Circ Cardiovasc Genet 2015; 8:427-436.

What is claimed is:

1. A method for assessing toxicity of a compound, comprising:
(1) detecting specific binding between the compound and DRP1 protein at its GTPase domain; and
(2) determining the compound as likely toxic.

2. The method of claim 1, wherein step (1) comprises simulating binding between the compound and the GTPase domain by molecular docking.

3. The method of claim 1, wherein step (1) comprises contacting the DRP1 protein or the GTPase domain of the DRP1 protein with the compound.

4. The method of claim 1, wherein step (1) comprises contacting a cell expressing the DRP1 protein with the compound.

5. The method of claim 1, further comprising, after step (2), performing an in vitro cell-based toxicity assay to assess cytotoxicity of the compound.

6. The method of claim 5, comprising measuring GTPase activity of the DRP1 protein in the presence and absence of the compound.

7. The method of claim 5, wherein an increase in the GTPase activity in the presence of the compound indicates toxicity of the compound.

8. The method of claim 5, comprising measuring polymerization of the DRP1 protein with itself or with binding partners FIS1, MFF, MIEF, MiD49, MiD51 in the presence and absence of the compound.

9. The method of claim 8, wherein an increase in the polymerization in the presence of the compound indicates toxicity of the compound.

10. The method of claim 5, comprising measuring localization of the DRP1 protein in the mitochondria in the presence and absence of the compound.

11. The method of claim 10, wherein an increase in the localization of DRP1 protein in the mitochondria in the presence of the compound indicates toxicity of the compound.

12. The method of claim 5, comprising measuring DRP1 expression level in the presence and absence of the compound.

13. The method of claim 12, wherein the expression level is DRP1 mRNA level.

14. The method of claim 12, wherein the expression level is DRP1 protein level.

15. The method of claim 12, wherein an increase in the DRP1 expression level in the presence of the compound indicates toxicity of the compound.

16. The method of claim 5, comprising monitoring morphology of mitochondria within the cell in the presence and absence of the compound.

17. The method of claim 16, wherein increased mitochondria fragmentation in the presence of the compound indicates toxicity of the compound.

18. The method of claim 5, comprising measuring redox potential of mitochondria within the cell in the presence and absence of the compound.

19. The method of claim 18, wherein reduced mitochondria redox potential in the presence of the compound indicates toxicity of the compound.

20. The method of claim 5, comprising measuring mRNA or protein level of one or more of ND1, ND5, COX6A2, and ATP6 within the cell in the presence and absence of the compound.

21. The method of claim 20, wherein a decrease in the mRNA or protein level of one or more of ND1, ND5, COX6A2, and ATP6 in the presence of the compound indicates toxicity of the compound

22. The method of claim 5, wherein the cell is a cardiomyocyte (CM), and wherein the method comprises monitoring morphology of sarcomere within the cell in the presence and absence of the compound.

23. The method of claim 22, wherein increased sarcomere disarray in the presence of the compound indicates toxicity of the compound.

24. The method of claim 5, wherein the cell is a human induced pluripotent stem cell-derived cardiomyocyte (hiPCS-CM).
